(19) United States
(12) Patent Application Publication
(10) Pub. No.: US 2009/0061024 A1
(43) Pub. Date: Mar. 5, 2009

(54) COMPOSITIONS AND METHODS EMPLOYING NMDA ANTAGONISTS FOR ACHIEVING AN ANESTHETIC-SPARING EFFECT

(75) Inventors: Cecil Mark Eppler, Langhome, PA (US); David Robert Hustead, Overland Park, KS (US); Thomas Gerard Cullen, Milltown, NJ (US); Raphael Johannes Zwijnenberg, Lambertville, NJ (US); William W. Muir, III, Columbus, OH (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/198,489

(22) Filed: Aug. 26, 2008

Related U.S. Application Data
(60) Provisional application No. 60/968,236, filed on Aug. 27, 2007.

(51) Int. Cl. A61K 33/00; A61K 31/675; A61P 23/00

(52) U.S. Cl. 424/718; 514/79

(57) ABSTRACT

Provided herein are compositions, combinations, and methods comprising NMDA antagonists including, but not limited to, NMDA glutamate receptor antagonists such as [2-(8,9-dioxo-2,6-diazabicyclo[5.2.0]non-1-(7)-en-2-yl)alkyl]phosphonic acid and derivatives thereof, which are effective in reducing the amount of anesthetic required to maintain anesthesia (i.e. to achieve an anesthetic-sparing effect).

### CROSS-REFERENCE TO RELATED APPLICATIONS

This application claims the benefit under 35 U.S.C. §119(e) to U.S. provisional application no. 60/968,236, filed Aug. 27, 2007, each of which is hereby incorporated by reference in its entirety.

### BACKGROUND OF THE DISCLOSURE

1. Technical Field of the Disclosure

The present disclosure relates generally to the field of medicine, including veterinary medicine. More specifically, the present disclosure provides compositions, combinations, kits and methods comprising NMDA glutamate receptor antagonists including, but not limited to, the compound: [2-(8,9-dioxo-2,6-diazabicyclo[5.2.0]non-1-(7)-en-2-yl)ethyl]phosphonic acid and derivatives thereof, which compounds, compositions, combinations kits and methods are effective for achieving an anesthetic-sparing effect.

2. Description of the Related Art

Anesthetic-sparing effects have been noted for several classes of drugs used to complement the beneficial effects, and/or mitigate undesirable side effects, of anesthetics. These so-called “anesthetic adjuvant” drugs include α-2 adrenergic agonists (Soares et al., American Journal of Veterinary Research 96:854-859 (2004) and Muir and Lerch, Am. J. Vet. Res. 67:782-789 (2006)), benzodiazepines (Hall et al., Anesthesiology 68:862-866 (1988)); and opioids (Machado et al., Veterinary Anesthesia and Analgesia 33:70-77 (2006) and Muir et al., Am. J. Vet. Res. 64:1-6 (2003)). Anesthetic sparing can also be achieved by blocking NMDA glutamate receptors. Ketamine, a non-competitive NMDA glutamate receptor antagonist, is commonly used as a hypnotic/dissociative/analgesic adjuvant for anesthetics. The anesthetic-sparing effects of 10-20% provided by ketamine at doses typically used clinically are rather modest (Muir et al., Am. J. Vet. Res. 64:1-6 (2003)), but are still considered one of the benefits of ketamine as an anesthetic adjuvant.

The anesthetic-sparing effects attainable through currently used anesthetic adjuvant drugs are limited by undesirable side effects, however. For example, the dissociative and other dysphoric effects of ketamine referenced above can persist into the post-surgical setting, where they are considered undesirable side-effects. Ketamine is often administered by IV infusion at relatively low doses rather than by a bolus IV injection (which would be more convenient) to avoid these side effects. Use-limiting side effects of other anesthetic adjuvant drugs include bradycardia for both α-2 adrenergic agonists (Salmenperra et al., Anesthesiology 80:837-846 (1994)) and opioids (Ilkiw et al., Canadian Journal of Veterinary Research 58:248-253 (1994)) and respiratory depression for opioids (van den Berg et al., British Journal of Clinical Pharmacology 38:533-543 (1994); Willette et al., Journal of Pharmacology and Experimental Therapeutics 240:352-358 (1987)). Although benzodiazepines can provide significant anesthetic-sparing effects, they tend to be rather modest (typically less than 25%) at doses used clinically (Tranquilli et al., American J. of Vet. Res. 52:662-664 (1991); Muir et al., Journal of Veterinary Pharmacology and Therapeutics 14:46-50 (1991)), reaching the approximately 50% level only at distinctly non-clinical doses (Hall et al., Anesthesiology 68:862-866 (1988)) where side effects such as respiratory depression and reduced analgesic efficacy of concurrently used opioids may occur (Gear et al., Pain 71:25-29 (1997) and Daghero et al., Anesthesiology 66:944-947 (1987)).

Glutamate and aspartate play dual roles in the central nervous system (CNS) as essential amino acids and as the principal excitatory neurotransmitters. There are at least four classes of excitatory amino acid receptors: NMDA (N-methyl-D-aspartate), AMPA (2-amino-3-(methyl-3-hydroxyisoxazol-4-yl)propanoic acid), kainate, and metabotropic receptors. These excitatory amino acid receptors regulate a wide range of signaling events that impact physiological brain functions. For example, activation of the NMDA receptor has been shown to be the central event that leads to excitotoxicity and neuronal death in many disease states, as well as a result of hypoxia and ischemia following head trauma, stroke, and following cardiac arrest. It is also known that the NMDA receptor plays a major role in the synaptic plasticity that underlies many higher cognitive functions, such as memory and learning, certain nociceptive pathways, and in the perception of pain. In addition, certain properties of NMDA receptors suggest that they may be involved in the information-processing in the brain that underlies consciousness itself (above information. (Reviewed in Petrenko et al., Anesth. Analg. 97:1108-1116 (2003)).

NMDA glutamate receptors (or “NMDA receptors”) are localized throughout the CNS and in nerves projecting from the CNS to peripheral tissues. NMDA receptors are ligand-gated cation channels that modulate sodium, potassium, and calcium ion flux when they are activated by glutamate in combination with glycine (reviewed by Childers and Baudy, Journal of Medicinal Chemistry 50:2557-2562 (2007)). Functional NMDA receptors are heterotetramers, consisting of 1-3 NR1 subunits and 1-3 NR2 subunits (generally depicted as 2 NR1+2 NR2). This heterogeneity is greatly augmented by the existence of at least 8 NR1 splice variants and 4 NR2 subunits (NR2A−NR2D). NR1 subunits, which can constitute ion channels when expressed alone, contain the glycine-binding site. NR2 subunits, which are necessary for full ion conductance, contain the glutamate-binding site and also allosteric modulatory sites for polyamines and Zn²⁺. The NMDA receptor also contains a Mg²⁺ binding site located inside the pore of the ion channel, which blocks ion flow through the channel when occupied by Mg²⁺.

Activation of NMDA receptors plays a major role in the induction of pain associated with peripheral tissue and nerve injury (Sindrup et al., Pain 83:389-400 (1999) and Salter, Cur. Topics in Med. Chem. 5:557-567 (2005)). Under conditions of normal (nociceptive) pain, the excitatory signal received from afferent neurons in the spinal cord dorsal horn is mediated primarily by the fast-inactivating kainate and AMPA subtypes of the glutamate receptor. Painful stimuli of greater duration and intensity result in accumulating, prolonged, slowly depolarizing synaptic potentials that relieve the NMDA subtype of the glutamate receptor from its tonic block by Mg²⁺ ions. Activation of NMDA receptors accentuates the sustained depolarization and contributes to an increase in the discharge of dorsal horn nociceptive neurons in a process called “wind-up.” Prolonged activation of NMDA receptors can lead to modifications in cellular signaling pathways that enhance the responsiveness of the nociceptive neuron to activation in a collection of processes referred to as “central sensitization.” The elements of central sensitization, such as reversible post-translational modification of proteins, may act over both the short term and longer term. Central sensitization includes both short-term, reversible components (such as post-translational modification of proteins) and long-term elements. One such long-term element thought to be associated with neuropathic pain is an enhanced response of the NMDA receptor itself to excitatory input through up-regulation of the modulatory tyrosine kinase Src. Yu and Salter, Proc. Natl. Acad. Sci. U.S.A. 96:7697-7704 (1999).

Earlier demonstrations that NMDA receptor antagonists could inhibit the “wind-up” response had provided the initial evidence for involvement of NMDA receptors in central sensitization and supported further efforts to develop novel analgesics targeting this mechanism. In basic studies with isolated nerve fibers and dorsal horn sensory neurons, various competitive and non-competitive NMDA receptor antagonists including D-CPP, d-APV, and MK-801 inhibited the cellular correlates of wind-up and central sensitization such as sustained depolarization and increased action potential discharge with repeated stimulation (Davies and Lodge, Brain Research 424:402-406 (1987); Dickenson and Sullivan, Neuropharmacology 26:1235-1238 (1987); and Woolf and Thompson, Pain 44(3):293-299 (1991)). Clinical studies with ketamine showed significant reductions of neuropathic and post-surgical pain (Eide et al., Pain 61:221-228 (1995); Roytblat et al., Anesth. Analg. 77:1161-1165 (1993); and Dich-Nielsen et al., Acta Anesthesiologica Scandinavica 6:538-587 (1992)).

NMDA receptor antagonists fall into several classes by mechanism, as expected given the structural complexity of NMDA receptors. NMDA receptor glutamate site antagonists refer to those compounds that interact competitively with the glutamate binding site of the NR2 subunit, for example CGS-19755 (Selfotel; cis-4-phosphonomethyl-2-piperidine carboxylic acid); CPP (3-(2-carboxypiperazinyl-4-yl)propyl-1-phosphonic acid); and AP5 (D-2 amino 5-phosphonopentanoic acid). See, e.g., Karlsten and Gordh, Drugs and Aging 11:398-412 (1997). Antagonists interacting at the strychnine-insensitive glycine site (glycine_{β}), for example L-701324 (7-chloro-4-hydroxy-3-(3-phenoxy)phenyl-2(1H)-quinoline), and blocking (or indirectly modulating) polyamine activation of NR2B-containing receptors, for example ifenprodil, have also been developed. Noncompetitive NMDA receptor channel-blocking antagonists include dizocilpine (MK-801), ketamine, dextromethorphan, memantine, and amantadine.

All of the compounds listed above have shown activity in preclinical pain models. See e.g., Hao et al., Pain 66:279-285 (1996); Bennett, J. Pain Symptom Management 19:S2 (2000); and Childers and Baudy, J. Med. Chem. 50:2557-2562 (2007). The noncompetitive channel blockers are the only class of NMDA receptor antagonists currently being used clinically for analgesia. Ketamine has shown efficacy for post-traumatic pain and allodynia (Max et al., Clinical Neuropharmacology 18:360-368 (1995); neuropathic pain (Leung et al., Pain 91:77-187 (2001) and Chizh and Hedley, Curr. Pharm. Design 11:2977-2994 (2005)); and postoperative pain (Slingsby and Waterman-Pearson, Res. Vet. Sci. 69:147-152 (2000) and DeKock et al., Pain 92:373-380 (2001)). Dextromethorphan has shown efficacy for treating diabetic neuropathy pain (Nelson et al., Neurology 48:1212-1218 (1997) and Sang et al.,. Anesthesiology 96:1053-1061 (2002)) and, with mixed success, for postoperative pain as an adjunct to opioids (Duedahl et al., Acta Anesthesiol. Scand. 50:1-13 (2006)). Amantadine has been used to treat postsurgical neuropathic pain in cancer patients (Pud et al., Pain 75:349-354 (1998)) and phantom limb pain (Wiech et al., Anesth. Analg. 98:408-413 (2004)).

Clinical usefulness of the noncompetitive channel-blocking NMDA antagonists has, however, been limited by adverse effects such as auditory and visual disturbances and hallucinations, feelings of unreality, feelings of detachment from the body, dizziness, sedation, nausea, and vomiting (Chizh and Hedley, Curr. Pharm. Design 11:2977-2994 (2005); Kohrs and Durieux, Anesth. Analg. 87:1186-1193 (1998); and Max et al., Clin. Neuropharm. 18:360-368 (1995)). Some of these effects are similar to those of phencyclidine (PCP), an abused psychotomimetic substance which interacts with the same site in the NMDA receptor (Javitt and Zukin, Am. J. Psychiatry 148:1-10 (1991) and Parsons et al., Drug News Perspect. 11:523-569 (1998)). Although it has been suggested that lower affinity channel blockers such as dextromethorphan, amantadine, and memantine might have fewer adverse effects than the high affinity blockers (Rogawski, Trends Pharmacol. Sci. 14:325 (1998)), the clinical efficacies of these drugs have been relatively modest with still problematic side effects (Nelson et al., Neurology 48:1212-1218 (1997); Sang et al.,. Anesthesiol. 96:1053-1061 (2002); Chizh and Hedley, Curr. Pharm. Design 11:2977-2994 (2005); and Sang, J. Pain and Symptom Management 19S:21-25 (2000)). Also, dizocilpine (very high affinity) and memantine (relatively low affinity) both substitute for the PCP-like discriminative stimulus effects in rats trained to distinguish between PCP and saline (Mori et al., Behav. Brain Res. 119:33-40 (2001)), and memantine has been shown to maintain PCP-like self-administration in monkeys, suggesting that it might have abuse potential in humans (Nicholson et al., Behav. Pharmacol. 9:231-243 (1998)).

Although NMDA receptor glutamate antagonists do not have the same degree of psychotomimetic side effects in humans or PCP-like discriminative stimulus effects in non-humans as the NMDA receptor channel blockers, they have been shown to have many undesirable side effects (Baron and Woods, Psychopharmacol. 118:42-51 (1995); Mori et al., Behav. Brain Res. 119:33-40 (2001); France et al., J. Pharm. Exp. Ther. 257:727-734 (1991); and France et al., Eur. J. Pharmacol. 159:133-139 (1989)). For example, the NMDA glutamate antagonist CGS-19755 has been shown to have a transient, reversible induction of vacuoles in some layers of the cingulate and retrosplenial cortices of mice and rats at behaviorally effective doses (i.e. effectiveness:vacuolization ratio of 1; Herring et al., “Excitatory Amino Acids Clinical Results with Antagonists,” (Academic Press, Chapter 1 (1997)). Although the functional implications of vacuolization are unclear, previous studies suggest that this vacuolization correlates with the psychotomimetic effects produced by NMDA receptor antagonists and might lead to limited neuronal cell death as in the case of dizocilpine (Olney et al., Science 244:1630-1632 (1989); Olney et al., Science 254:1515-1518 (1991); and Fix et al., Exp. Neurol. 123:204-215 (1993)).

U.S. Pat. No. 5,168,103 to Kinney et al. (“the '103 patent”) discloses certain [[2-(Amino-3,4-dioxo-1-cyclobuten-1-yl)amino]alkyl]-acid derivatives useful as neuroprotectant and anticonvulsant agents. These [[2-(Amino-3,4-dioxo-1-cyclobuten-1-yl)amino]alkyl]-acid derivatives are disclosed as competitive NMDA antagonists useful to treat certain central nervous system disorders such as convulsions, brain cell damage, and related neurodegenerative disorders. Side effects of one of the compounds disclosed in the '103 patent, [2-(8,9-dioxo-2,6-diazabicyclo[5.2.0]non-1(7-en-2-yl)ethyl]phosphonic acid (a/k/a perzinfotel and EAA-090) were evaluated in healthy human volunteers in a phase I clinical study in Europe, done in connection with developing the compound for treating stroke-related ischemia in patients (Bradford et al., Stroke and Cerebral Circulation, Abstract (1998).

U.S. Pat. No. 7,098,200 to Brandt et al. (the '200 patent) discloses that perzinfotel is effective in producing antihyperalgesic effects in a variety of preclinical pain models. For example, perzinfotel produced antihyperalgesic effects under conditions in which comparator NMDA receptor antagonists did not. Additionally, perzinfotel did not have the degree of adverse side effects exhibited by known NMDA receptor antagonists at dosages needed to produce antihyperalgesic effects. For example, perzinfotel did not produce ataxia or sedation in comparison to other reported competitive glutamate antagonists (CGS-19755), competitive polyamine antagonists (ifenprodil) and use dependent channel blockers (MK-801, memantine; dizocilipine, ketamine) at doses needed to relieve hyperalgesia in preclinical pain models.

Additionally, some NMDA receptor antagonists, such as CGS-19755 have been found to exhibit a transient, reversible induction of vacuoles in some layers of the cingulate and retrosplenial cortices of mice and rats. In contrast to CGS-19755, which caused vacuolization at behaviorally effective doses, perzinfotel had an effectiveness:vacuolization ratio as large as 16. Moreover, unlike the NMDA receptor channel blocking antagonists, perzinfotel did not substitute for PCP in rats, suggesting that this compound would not be associated with PCP-like psychotomimetic effects or contain PCP-like abuse liability. Additionally, perzinfotel was devoid of many PCP-like effects up to doses 4-10 times higher than those effective in an ischemia model.

Perzinfotel has been described as a potent, selective, competitive NMDA antagonist that exhibits a superior therapeutic index for efficacy versus psychotomimetic side effects (Childers et al., Drugs of the Future 27:633-638 (2002)). Perzinfotel possesses a bioisosteric squaric acid amide in place of the typical α-amino acid and is reported to be 10-fold selective for rodent NMDA receptors possessing the NR2A subunit (Sun et al., J. Pharm. Exp. Ther. 310:563-570 (2004)). Perzinfotel has demonstrated efficacy in animal models of inflammatory pain when administered both intraperitonealy and orally (Brandt et al., J. Pharm. Exp. Ther. 313:1379-1386 (2005)).

U.S. Patent Publication No. 2006/0079679 to Baudy (the '679 publication) discloses useful derivatives of perzinfotel, such as diethyl 3,3′-[({2-[8,9-dioxo-2,6-diazabicyclo[5.2.0]non-1(7)-en-2-yl]ethyl}phosphoryl)bis(oxy)] dibenzoate and derivatives thereof. These compounds function as “prodrugs,” providing improved oral absorption relative to perzinfotel (due to increased lipophilicity) and yielding perzinfotel in vivo upon hydrolysis by plasma esterases.

Although isoflurane-sparing effects have been shown preclinically (in rats) for the competitive NMDA antagonists CPP and CGS-19755 (Kuroda et al., Anesth. Analg. 77:795-800 (1993)), clinical use is unlikely due to unacceptable side effects documented above and also by Hoyte et al. (Current Molecular Medicine 4:131-136 (2004)) and Childers and Baudy (J. Med. Chem. 50:2557-2562 (2007)). Thus, there remains a need in the art for compositions and methods, including compositions and methods employing NMDA antagonists such as perzinfotel and derivatives thereof, for achieving improved anesthetic-sparing effects while exhibiting reduced undesirable side effects.

### SUMMARY OF THE DISCLOSURE

The present disclosure fulfills these and other related needs by providing compositions, combinations, and methods comprising NMDA glutamate receptor antagonists including, but not limited to, [2-(8,9-dioxo-2,6-diazabicyclo[5.2.0]non-1-(7)-en-2-yl)alkyl]phosphonic acid (perzinfotel) and derivatives thereof, which are effective in mediating surprisingly robust anesthetic-sparing effects while also providing the surprising additional benefit of improved cardiopulmonary function relative to the anesthetic alone. That is, compositions and methods disclosed herein, when used in conjunction with an anesthesia regimen, permit the use of a reduced concentration of anesthetic than would otherwise be required in the absence of the NMDA receptor antagonist, to achieve an equivalent level of anesthesia. Such an anesthetic-sparing effect is exemplified herein by the NMDA glutamate receptor antagonist perzinfotel, and derivatives thereof such as, for example, diethyl 3,3′-[({2-[8,9-dioxo-2,6-diazabicyclo[5.2.0]non-1(7)-en-2-yl]ethyl}phosphoryl)bis(oxy)] dibenzoate. These compounds have been disclosed and described in U.S. Pat. Nos. 5,168,103 and 7,098,200 and U.S. Patent Publication No. 2006/0079679, which are incorporated herein by reference in their entireties.

As disclosed in further detail herein, the NMDA glutamate receptor antagonist perzinfotel is capable of producing substantial anesthetic-sparing effects when used in combination with anesthetics, exemplified herein, but not limited to, isoflurane. More specifically, it is demonstrated that perzinfotel gives anesthetic-sparing effects of up to about 60% at doses in which reduced cardiopulmonary function is not observed. In fact, within certain embodiments, the NMDA receptor antagonist:anesthetic combinations, for example the perzinfotel:isoflurane combination exemplified herein, exhibit improved cardiopulmonary function as compared to effects achieved with the anesthetic alone.

NMDA antagonists presented herein may be administered during surgical procedures to allow effective anesthesia to be produced by reduced amounts of anesthetic compounds including, but not limited to, isoflurane. The safety of surgical procedures is improved due to lower concentrations of anesthetic required, which results in reduced deleterious effects on the homeostatic mechanisms regulating cardiopulmonary and other functions as well as the bispectral index, a measure of depth of unconsciousness derived from electroencephalograph data, which is either unchanged or increased (toward increased consciousness) relative to anesthetic alone when concentrations of perzinfotel and derivatives thereof are employed to achieve an anesthetic-sparing effect.

These and other embodiments, features, and advantages of the invention will become apparent from the detailed description and the appended claims set forth herein below.

### DETAILED DESCRIPTION OF THE DISCLOSURE

As indicated above, the present disclosure is based upon the unexpected discovery that certain NMDA glutamate receptor antagonists, including perzinfotel, and derivatives thereof, are capable of producing substantial anesthetic-sparing effects when used in combination with anesthetics such as, for example, isoflurane. That is, when administered during a surgical procedure, perzinfotel allows effective anesthesia to be achieved with reduced amounts of an anesthetic compound. Perzinfotel gives anesthetic-sparing effects of between about 13% and about 59%, with improved cardiopulmonary function relative to anesthetic alone at doses required to produce equivalent levels of anesthesia.

The present invention will be best understood by reference to the following definitions:

### Definitions

As used herein, the term “alkyl” refers to an aliphatic hydrocarbon chain having 1 to 12 carbon atoms and includes, but is not limited to, straight or branched chains, such as methyl, ethyl, n-propyl, isopropyl, n-butyl, isobutyl, sec-butyl, t-butyl, n-pentyl, isopentyl, neo-pentyl, n-hexyl, and isohexyl. Lower alkyl refers to alkyl having 1 to 3 carbon atoms. In some embodiments of the invention, alkyl is preferably C₁ to C₈ and, more preferably, C₁ to C₆.

As used herein, the term “alkylenyl” refers to a linking alkyl group (or bivalent alkyl group), for example, ——CH₂—— or ——(CH₂)₂——.

As used herein, the term “alkenyl” refers to an aliphatic straight or branched hydrocarbon chain having 2 to 7 carbon atoms that contains 1 to 3 double bonds. Examples of alkenyl are straight or branched mono-, di-, or poly-unsaturated groups, such as vinyl, prop-1-enyl, allyl, methallyl, but-1-enyl, but-2-enyl or but-3-enyl.

As used herein, the term “alkenylenyl” refers to a linking alkenyl group (or a bivalent alkenyl group), for example, ——CH═CH——.

As used herein, the term “alkynyl” refers to an aliphatic, straight or branched, hydrocarbon chain having 2 to 7 carbon atoms that may contain 1 to 3 triple bonds.

As used herein, the term “acyl” refers to the group R——C(═O)—— wherein R is an alkyl group of 1 to 6 carbon atoms. For example, a C₂ to C₇ acyl group refers to the group R——C(═O)—— where R is an alkyl group of 1 to 6 carbon atoms.

As used herein, the term “alkanesulfonyl” refers to the group R——S(O)₂—— wherein R is an alkyl group of 1 to 6 carbon atoms.

As used herein, the term “aryl” refers to an aromatic 5- to 13-member mono- or bi-carbocyclic ring, such as phenyl or naphthyl. Groups containing aryl moieties may be monocyclic having 5 to 7 carbon atoms in the ring. Heteroaryl means an aromatic 5- to 13-membered, carbon containing, mono- or bi-cyclic ring having one to five heteroatoms that, independently, may be selected from nitrogen, oxygen, and sulfur. Groups containing heteroaryl moieties may be monocyclic having 5 to 7 members in the ring where one to two of the ring members are selected, independently, from nitrogen, oxygen or sulfur. Groups containing aryl or heteroaryl moieties may optionally be substituted as defined below or unsubstituted.

As used herein, the term “aroyl” refers to the group Ar——C(═O)—— where Ar is aryl as defined above. For example, a C₆ to C₁₄ aroyl moiety refers to the group Ar——C(═O)—— where Ar is an aromatic 5 to 13 membered carbocyclic ring.

As used herein, the term “halogen” refers to fluorine, chlorine, bromine, or iodine.

As used herein, the term “substituted” refers to a moiety, such as an aryl or heteroaryl moiety, having from 1 to about 5 substituents and/or from 1 to about 3 substituents, independently selected from the group consisting of halogen, cyano, nitro, hydroxyl, C₁-C₆ alkyl, and C₁-C₆ alkoxy. Substituents may be halogen, hydroxyl, or C₁-C₆ alkyl.

As used herein, the terms “subject” or “animal” refer, interchangeably, to vertebrates including, but not limited to, members of the mammalian species, such as canine, feline, lupine, mustela, rodent (e.g., racine and murine, etc.), equine, bovine, ovine, caprine, porcine species, and primates, the latter including humans.

As used herein, the phrase “pharmaceutically acceptable” refers to substances that are acceptable for use in pharmaceutical applications from a toxicological perspective and does not adversely interact with the active ingredient. “Pharmaceutically acceptable” includes molecular entities and compositions that are physiologically tolerable and do not typically produce an allergic or similar untoward reaction, such as gastric upset, dizziness, and the like, when administered to a subject. The term “pharmaceutically acceptable” may include molecular entities and compositions that are approved by a regulatory agency of the Federal or a state government or listed in the U.S. Pharmacopeia or other generally recognized pharmacopeia for use in animals, and more particularly in humans.

The compounds useful in the anesthetic-sparing compositions and methods of the present disclosure also include pharmaceutically acceptable salts of the NMDA glutamate receptor antagonists presented herein. By “pharmaceutically acceptable salt” is meant any compound formed by the addition of a pharmaceutically acceptable base or acid to a compound presented herein to form the corresponding salt. Preferably, the pharmaceutically acceptable salts are alkali metal (sodium, potassium, or lithium) or alkaline earth metal (calcium or magnesium) salts of the presently disclosed compounds, or salts of the compounds with pharmaceutically acceptable cations derived from ammonia or a basic amine. Examples of the latter include, but are not limited to, ammonium, mono-, di-, or trimethylammonium, mono-, di-, or triethylammonium, mono-, di-, or tripropylammonium (iso and normal), ethyldimethylammonium, benzyldimethylammonium, cyclohexylammonium, benzylammonium, dibenzylammonium, piperidinium, morpholinium, pyrrolidinium, piperazinium, 1-methylpiperidinium, 1-isopropylpyrrolidinium, 1,4-dimethylpiperazinium, 1-n-butylpiperidinium, 2-methylpiperidinium, 1-ethyl-2-methylpiperidinium, mono-, di-, or triethanolammonium, tris-(hydroxymethyl)methylammonium, or phenylmonoethanolammonium.

The term “carrier” refers to a diluent, adjuvant, excipient, or vehicle with which the compound is administered. Such carriers can be sterile liquids, such as water and oils, including those of petroleum, animal, vegetable, or synthetic origin, such as peanut oil, soybean oil, mineral oil, sesame oil, and the like. Water or aqueous solution saline solutions and aqueous dextrose and glycerol solutions are preferably employed as carriers, particularly for injectable solutions. Suitable carriers are described in “Remington's Pharmaceutical Sciences” by E. W. Martin, 18^{th} Edition.

In a specific embodiment, the term “about” or “approximately” means within a statistically meaningful range of a value. Depending upon the precise application contemplated, such a range can be within 20%, or within 10%, or within 5% of a given value or range. The allowable variation encompassed by the term “about” or “approximately” depends on the particular system under study, and can be readily appreciated by one of ordinary skill in the art.

The term “subject” as used herein includes human and non-human animals, such as dogs, cats, cattle, sheep, horses, goats, pigs, llamas, camels, water buffalo, donkeys, rabbits, fallow deer, reindeer, minks, chinchillas, ferrets, raccoons, chickens, geese, turkeys, ducks and the like.

One embodiment of the invention provides a method for achieving an anesthetic-sparing effect in a subject, said method comprising administering to said subject an NMDA glutamate receptor antagonist and a general anesthetic;

wherein an anesthetic-sparing effect is achieved in the subject.

Another embodiment of the invention provides a method for anesthetizing a subject comprising: administering to the subject an NMDA glutamate receptor antagonist and a general anesthetic.

Another embodiment provides the use of an NMDA glutamate receptor antagonist in combination with a general anesthetic for achieving an anesthetic-sparing effect in a subject. Another embodiment provides the use of an NMDA glutamate receptor antagonist in combination with a general anesthetic for prolonging anesthesia in a subject.

Another embodiment provides the use of an NMDA glutumate receptor antagonist in the manufacture of a medicament for combination therapy by simultaneous, separate or sequential administration with a general anesthetic, for achieving an anesthetic sparing effect in a subject.

In another embodiment of any of the embodiments described herein, the general anesthetic is administered before administration of the NMDA glutamate receptor antagonist. Alternatively, the general anesthetic is administered during or after administration of the NMDA glutamate receptor antagonist.

Preferably, the NMDA glutamate receptor antagonist is [2-(8,9-dioxo-2,6-diazabicyclo[5.2.0]non-1-(7)-en-2-yl)ethyl]phosphonic acid or a tautomer or pharmaceutically acceptable salt thereof

In another embodiment, said NMDA glutamate receptor antagonist is a compound of formula (I) or a pharmaceutically acceptable salt or tautomer thereof:

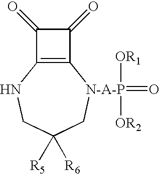

wherein A is alkylenyl of 1 to 4 carbon atoms;
R₁ and R₂ are, independently, hydrogen or phenyl optionally substituted with 1 to 2 substituents, independently, selected from the group consisting of —C(O)R₃, halogen, cyano, nitro, hydroxyl, C₁-C₆ alkyl, and C₁-C₆ alkoxy;
R₃ is, independently, hydrogen, —OR₄, alkyl, aryl, or heteroaryl;
R₄ is hydrogen, alkyl, aryl, or heteroaryl;
R₅ and R₆ are, independently, hydrogen, alkyl, hydroxyl, alkoxy, or phenyl;
wherein any R₃ to R₆ group having an aryl or heteroaryl moiety can optionally be substituted on the aryl or heteroaryl moiety with 1 to about 5 substituents, independently, selected from the group consisting of halogen, cyano, nitro, hydroxyl, C₁-C₆ alkyl, and C₁-C₆ alkoxy.

More particularly, said NMDA glutamate receptor antagonist is [2-(8,9-dioxo-2,6-diazabicyclo[5.2.0]non-1-(7)-en-2-yl)ethyl]phosphonic acid or diethyl 3,3′-[({2-[8,9-dioxo-2,6-diazabicyclo[5.2.0]non-1(7)-en-2-yl]ethyl}phosphoryl)bis(oxy)] dibenzoate or a pharmaceutically acceptable salt thereof.

In another embodiment, said general anesthetic is administered via inhalation or intravenously. In another embodiment, said NMDA glutamate receptor antagonist is administered parenterally (i.e. subcutaneously, intravenously, intramuscularly, intrasternaly, or by infusion techniques).

Another embodiment further comprises administering an additional anesthetic agent. In another embodiment, said additional or general anesthetic is selected from the group consisting of ketamine, thiopental, methohexital, etomidate, propofol, flumazenil, retamine, remifentanyl, midazolam, pentothal, and evipal procaine. More particularly, the general anesthetic is isoflurane and the additional anesthetic agent is propofol. In another embodiment, said general anesthetic is selected from the group consisting of halothane, isoflurane, sevoflurane, desflurane, ethylene, cyclopropane, ether, chloroform, nitrous oxide, and xenon. More particularly, said general anesthetic is isoflurane.

Another embodiment further comprises the step of administering to said subject one or more pharmaceutically active agent selected from the group consisting of an analgesic agent, a muscle-relaxing agent, and a hypnotic/dissociative agent.

Another embodiment further comprises the step of administering to said subject one or more pharmaceutically active agent selected from the group consisting of a benzodiazepine, an opioid, an α-2 adrenergic agonist, a non-steroidal anti-inflammatory drug (NSAID), a corticosteroid, a barbiturate, a non-barbiturate hypnotic a dissociative, a channel-blocking NMDA antagonist, and an injectable. In another embodiment, said benzodiazepine is zolazepam or valium. In another embodiment, said opioid is morphine, butorphanol or fentanyl. In another embodiment, said α-2 adrenergic agonist is medetomidine or xylazine. In another embodiment, said NSAID is etodolac, carprofen, deracoxib, firocoxib, tepoxalin, or meloxicam. In another embodiment, said corticosteroid is cortisol. In another embodiment, said barbiturate is phenobarbital or thiopental. In another embodiment, said non-barbiturate hypnotic is etomidate or alphaxan. In another embodiment, said channel-blocking NMDA antagonist is ketamine or tiletamine. In another embodiment, said injectable is propofol or alfaxan.

In a preferred embodiment of the present invention, said subject is a dog, cat, horse, cattle, or pig.

Another embodiment of the present invention provides a method for prolonging anesthesia in a subject comprising, administering to the subject [2-(8,9-dioxo-2,6-diazabicyclo[5.2.0]non-1-(7)-en-2-yl)ethyl]phosphonic acid or a pharmaceutically acceptable salt thereof and a general anesthetic. In a more particular embodiment, the general anesthetic is administered before administration of [2-(8,9-dioxo-2,6-diazabicyclo[5.2.0]non-1-(7)-en-2-yl)ethyl]phosphonic acid or a pharmaceutically acceptable salt thereof. In another embodiment, the general anesthetic is administered during or after administration of [2-(8,9-dioxo-2,6-diazabicyclo[5.2.0]non-1-(7)-en-2-yl)ethyl]phosphonic acid or a pharmaceutically acceptable salt thereof.

Another embodiment of the present invention provides a kit comprising an NMDA glutamate receptor antagonist and a general anesthetic. In a more particular embodiment, said NMDA glutamate receptor antagonist is [2-(8,9-dioxo-2,6-diazabicyclo[5.2.0]non-1-(7)-en-2-yl)ethyl]phosphonic acid or a pharmaceutically acceptable salt thereof. More particular still, the kit further comprises an additional anesthetic agent. More particular still, the general anesthetic is isoflurane and the additional anesthetic is propofol.

Another embodiment of the present invention provides for the preparation of a medicament comprising an NMDA glutamate receptor antagonist in combination with a general anesthetic for achieving an anesthetic-sparing effect in a subject. Another embodiment provides for the preparation of a medicament comprising NMDA glutamate receptor antagonists for achieving an anesthetic-sparing effect in combination with a general anesthetic in a subject.

Another embodiment of the invention provides for a composition comprising an NMDA glutamate receptor antagonist and a general anesthetic. The NMDA glutamate receptor antagonist and a general anesthetic can be in separate containers or in admixture.

### The NMDA Glutamate Receptor Antagonist [2-(8,9-dioxo-2,6-diazabicyclo[5.2.0]non-1-(7)-en-2-yl)ethyl]phosphonic acid (Perzinfotel) and Derivatives Thereof

As indicated above, the present invention is based upon the discovery that administration of an NMDA glutamate receptor antagonist, exemplified by perzinfotel, along with (i.e. before, simultaneously, or after) an anesthetic such as, for example, isoflurane, so that perzinfotel and the anesthetic are simultaneously effective, permits the maintenance of anesthesia at minimum alveolar concentrations (MACs) of anesthetic that are substantially reduced as compared to the MACs of anesthetic required in the absence of the NMDA glutamate receptor antagonist. It will be appreciated that this anesthetic-sparing effect may be achieved by additional or alternative NMDA glutamate receptor antagonists including, but not limited to, various derivatives of the NMDA glutamate receptor antagonist perzinfotel.

An exemplary NMDA glutamate receptor antagonist provided herein is “Perzinfotel” (EAA-090), which is: [2-(8,9-dioxo-2,6-diazabicyclo[5.2.0]non-1-(7)-en-2-yl)ethyl]phosphonic and is represented by the following formula:

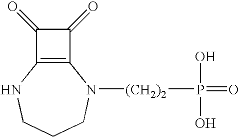

As indicated above, derivatives of NMDA glutamate receptor antagonists such as [2-(8,9-dioxo-2,6-diazabicyclo[5.2.0]non-1-(7)-en-2-yl)alkyl]phosphonic acid are disclosed in U.S. Patent Publication No. 2006/0079679, filed Oct. 6, 2005, which publication is incorporated herein by reference in its entirety.

Within certain embodiments, these derivatives of the NMDA glutamate receptor antagonist [2-(8,9-dioxo-2,6-diazabicyclo[5.2.0]non-1-(7)-en-2-yl)alkyl]phosphonic acid are represented by compounds of the following formula (I) or pharmaceutically acceptable salts thereof:

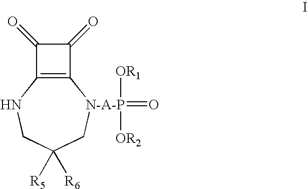

wherein A is alkylenyl of 1 to 4 carbon atoms, or alkenylenyl of 2 to 4 carbon atoms;
R₁ and R₂ are, independently, hydrogen or a C₅ to C₇ aryl optionally substituted with 1 to 2 substituents, independently selected from the group consisting of ——C(O)R₃, halogen, cyano, nitro, hydroxyl, C₁-C₆ alkyl, and C₁-C₆ alkoxy;
R₃ is hydrogen, —OR₄, alkyl, aryl, or heteroaryl;
R₄ is hydrogen, alkyl, aryl, or heteroaryl;
R₅ and R₆ are, independently, hydrogen, alkyl, hydroxyl, alkoxy, or C₅ to C₇ aryl;
wherein any R₃ to R₆ group having an aryl or heteroaryl moiety can optionally be substituted on the aryl or heteroaryl moiety with 1 to about 5 substituents independently selected from the group consisting of halogen, cyano, nitro, hydroxyl, C₁-C₆ alkyl, and C₁-C₆ alkoxy.

In another embodiment of the compound of formula (I):

A is alkylenyl of 1 to 4 carbon atoms;
R₁ and R₂ are, independently, hydrogen or phenyl optionally substituted with 1 to 2 substituents, independently, selected from the group consisting of —C(O)R₃, halogen, cyano, nitro, hydroxyl, C₁-C₆ alkyl, and C₁-C₆ alkoxy;
R₃ is, independently; hydrogen, —OR₄, alkyl, aryl, or heteroaryl;
R₄ is hydrogen, alkyl, aryl, or heteroaryl;
R₅ and R₆ are, independently, hydrogen, alkyl, hydroxyl, alkoxy, or phenyl;
wherein any R₃ to R₆ group having an aryl or heteroaryl moiety can optionally be substituted on the aryl or heteroaryl moiety with 1 to about 5 substituents, independently, selected from the group consisting of halogen, cyano, nitro, hydroxyl, C₁-C₆ alkyl, and C₁-C₆ alkoxy.

Within other embodiments, derivatives of the NMDA glutamate receptor antagonist [2-(8,9-dioxo-2,6-diazabicyclo[5.2.0]non-1-(7)-en-2-yl)alkyl]phosphonic acid are represented by compounds of the following formula (II) or pharmaceutically acceptable salts thereof:

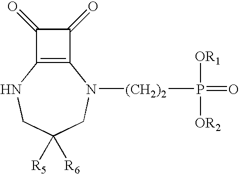

wherein, R₁ and R₂ are, independently, hydrogen or

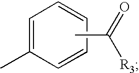

R₃ is hydrogen, —OR₄, alkyl, aryl, or heteroaryl,
R₄ is hydrogen, alkyl, aryl, or heteroaryl,
R₅ and R₆ are, independently, hydrogen, alkyl, OH, alkoxy, or C₅ to C₇ aryl;
wherein any R₃ to R₆ group having an aryl or heteroaryl moiety may optionally be substituted on the aryl or heteroaryl moiety with 1 to about 5 substituents independently selected from the group consisting of halogen, cyano, nitro, hydroxyl, C₁-C₆ alkyl, and C₁-C₆ alkoxy.

Within further embodiments, derivatives of the NMDA glutamate receptor antagonist [2-(8,9-dioxo-2,6-diazabicyclo[5.2.0]non-1-(7)-en-2-yl)alkyl]phosphonic acid are represented by compounds of the following formula (III) or pharmaceutically acceptable salts thereof:

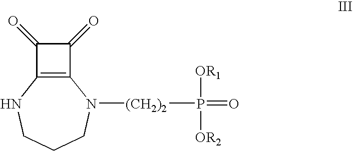

wherein
R₁ and R₂ are, independently, hydrogen or

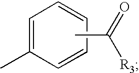

with the proviso that at least one of R₁ and R₂ is not hydrogen;
R₃ is hydrogen, alkyl, aryl, or heteroaryl; and
wherein any aryl or heteroaryl moiety may optionally be substituted on the aryl or heteroaryl moiety with 1 to about 5 substituents independently selected from the group consisting of halogen, cyano, nitro, hydroxyl, C₁-C₆ alkyl, and C₁-C₆ alkoxy.

Within yet further embodiments, derivatives of the NMDA glutamate receptor antagonist [2-(8,9-dioxo-2,6-diazabicyclo[5.2.0]non-1-(7)-en-2-yl)alkyl]phosphonic acid are represented by compounds of the following formula (III) or pharmaceutically acceptable salts thereof:

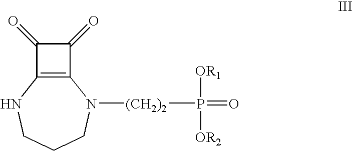

wherein
R₁ and R₂ are, independently, hydrogen or

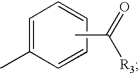

R₃ is —OR₄;
R₄ is hydrogen, alkyl, aryl, or heteroaryl; and
wherein any aryl or heteroaryl moiety may optionally be substituted on the aryl or heteroaryl moiety with 1 to about 5 substituents independently selected from the group consisting of halogen, cyano, nitro, hydroxyl, C₁-C₆ alkyl, and C₁-C₆ alkoxy.

In still further embodiments, the present disclosure provides compositions comprising at least one compound of the formula (I), (II), or (III), and pharmaceutically acceptable salts thereof, described above. In another embodiment of any of the foregoing compounds of formula (I), (II), or (III), at least one of R₁ and R₂ is not hydrogen.

### Methodology for the Synthesis of the NMDA Glutamate Receptor Antagonist [2-(8,9-dioxo-2,6-diazabicyclo [5.2.0]non-1(7)-en-2-yl)alkyl]phosphonic acid (perzinfotel) and Derivatives Thereof

Methodology for the synthesis of the NMDA glutamate receptor antagonist [2-(8,9-dioxo-2,6-diazabicyclo[5.2.0]non-1(7)-en-2-yl)alkyl]phosphonic acid and the derivatives and intermediates disclosed herein are presented in detail in U.S. Pat. Nos. 5,168,103, 5,990,307, and 6,011,168; in U.S. Patent Publication No. 2006/0079679; and in Synthetic Communications, 20(16):2559-2564 (1990) which are incorporated by reference herein in their entireties.

Schemes 1, 2 and 3 depict stems in the synthesis of [2-(8,9-dioxo-2,6-diazabicyclo[5.2.0]non-1(7)-en-2-yl)ethyl]phosphonic acid. Scheme 1 depicts the preparation of [2-(8,9-dioxo-2,6-diazabicyclo[5.2.0]non-1(7)-en-yl)alkyl]phosphonic by the following five-step protocol:

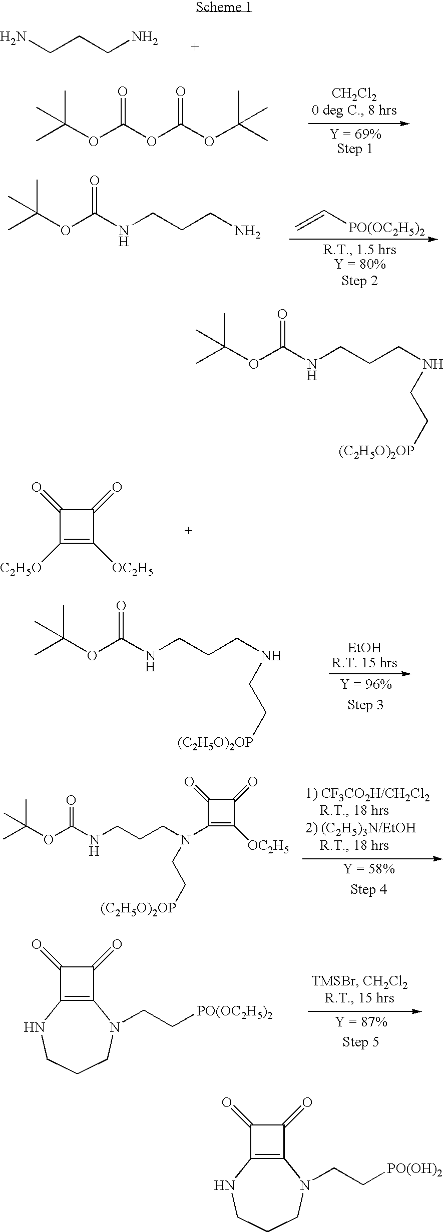

3-(t-butoxycarbonylamino)propaneamine (“t-BOC-propaneamine”)

A solution of di-t-butylcarbonate (50.1 g, 0.23 mole) in 200 mL methyl t-butyl ether (MTBE) is added dropwise over a period of three hours to a solution of 1,3-diaminopropane (83 g, 1.12 mole) in 600 mL methyl t-butyl ether (MTBE) and cooled to below 25° C. The mixture is allowed to stir for 22 hours at room temperature and the solvent removed under reduced pressure to generate an oil. Water (1000 mL) is added to the residue and the insoluble bis-substituted product ((3-tert-butoxycarbonylamino-propyl)carbamic acid tert-butyl ester) is removed by filtration. To the filtrate is added sodium chloride (5 grams). The filtrate is extracted with MTBE (5×150 mL). The combined organics are washed with saturated sodium chloride (1×25 mL), dried over sodium sulfate and concentrated to yield t-BOC-propaneamine (28.1 g) in a 69% yield. NMR (DMSO-d₆, 400 Mhz): 1.30 (s, 2H)), 1.45 (s, 9H), 1.5-1.65 (m, 2H), 2.74 (t, 2H), 3.25 (q, 2H), 4.95 (bs), 1H).

N-[3-(t-butyloxycarbonylamino)propyl]-2-aminoethylphosphonic acid diethyl ester

To a solution of 3-(t-butoxycarbonylamino)propaneamine (77 g, 0.44 mole) in methanol (500 mL) is added diethyl vinylphosphonate 97% (75 g, 0.44 mole) under nitrogen kept in a water bath at ˜20° C. for 48 hr. The reaction mixture is concentrated under reduced pressure and the residue (˜160 g) is placed on a pad of “Florosil” (3″×6″) and eluted with methylene chloride:hexane 1:1, then methylene chloride, and finally methylene chloride:methanol 9:1 to give N-[3-(t-butyloxycarbonylamino)propyl]-2-aminoethylphosphonic acid diethyl ester as a colorless oil (121 g, 80%). NMR (CDCl₃, 400 Mhz): 1.32 (t, 6H)), 1.43 (s, 9H), 1.65 (t, 2H) 1.80 (br, 1H), 1.97 (dt, 2H), 2.67 (t, 2H), 2.85 (dt, 2H), 3.20 (q, 2H), 4.09 (m, 4H), 5.08 (br, 1H).

N-[3-(t-butoxycarbonylamino)propyl]-N-[4-ethoxy-2,3-dioxocyclobut-1-ene-1-yl]-2-aminoethylphosphonic acid diethyl ester

To a solution of 3,4-diethoxy-3-cyclobutene-1,2-dione (45 g, 0.265 mole) in methanol (1.2 L) under nitrogen is added, dropwise, a solution of N-[3-(t-butyloxycarbonylamino)propyl]-2-aminoethylphosphonic acid diethyl ester (80 g, 0.24 mole) in methanol (600 mL) and the reaction mixture is stirred at room temperature for 15 hours. Thin layer chromatography (silica gel 60 F-254 (0.25 mm thickness) plates (visualization with UV light and/or iodine vapor) 89% methylene chloride, 10% methanol, and 1% ammonium hydroxide) shows that the reaction is complete. The reaction mixture is concentrated under reduced pressure and toluene (100 mL) is added and then removed under reduced pressure to N-[3-(t-butoxycarbonylamino)propyl]-N-[4-ethoxy-2,3-dioxocyclobut-1-ene-1-yl]-2-aminoethylphosphonic acid diethyl ester as a viscous oil (117 g, 96%). NMR (CDCl₃, 400 Mhz): 1.34 (t, 6H)), 1.43 (s, 9H), 1.46 (t, 3H) 2.12 (m, 2H), 3.14 (m, 2H), 3.49 (t, 1H), 3.66 (m, 1H), 3.73 (t, 1H), 3.90 (m, 1H), 4.10 (m, 4H), 4.74 (m, 4H), 5.05 (br, 1H).

[2-(8,9-dioxo-2,6-diazabicyclo[5.2.0]non-1(7)-en-2-yl)ethyl]phosphonic acid diethyl ester

A solution of N-[3-(t-butoxycarbonylamino)propyl]-N-[4-ethoxy-2,3-dioxocyclobut-1-ene-1-yl]-2-aminoethylphosphonic acid diethyl ester (100 g, 0.22 mole) in toluene (500 mL) is cooled in ice and treated with trifluoroacetic acid (300 mL). The reaction mixture is left to warm to ambient temperature overnight. The solution is concentrated under reduced pressure at a maximum temperature 40° C. Toluene is added (2×100 mL) and the solution concentrated to give a viscous oil (159.5 g). The viscous oil is dissolved in methanol and added dropwise over eight hours to a solution of triethylamine (350 mL) in methanol (1.5 L) and stirred for eight hours at room temperature. The reaction mixture is concentrated under reduced pressure to an oil which is taken up in ethyl acetate (1 L). The compound is crystallized and cooled on ice, filtered, and washed first with ethyl acetate and finally with hexane to give the title compound as a white compound (40 g, 58%). NMR (CDCl₃, 400 Mhz): 1.34 (t, 6H)), 2.06 (m, 2H), 2.20 (dt, 2H), 3.50 (m, 4H), 4.05 (m, 2H), 4.15 (m, 4H), 7.87 (br 1H).). MS (DEI) M⁺ m/z 316. LC analysis (column: Microsorb-MV C-18, 150×4.6 mm: Eluent 30/70 MeOH/0.01 M NH₄H₂PO₄ pH 4.7; Flow rate: 1 mL/min; UV detector at 210 nm; Analysis Calc'd for C₁₃H₂₁N₂O₅P: C, 49.36; H, 6.69; N, 8.85%; Found: C, 49.476; H, 6.74; N, 8.77%.

[2-(8,9-dioxo-2,6-diazabicyclo[5.2.0]non-1(7)-en-2-yl)ethyl]phosphonic Acid

[2-(8,9-dioxo-2,6-diazabicyclo[5.2.0]non-1(7)-en-2-yl)alkyl]phosphonic acid is prepared as follows. Under a nitrogen atmosphere, bromotrimethylsilane (83 mL, 96.3 g, 0.63 mole) is added dropwise at a fast rate to a solution of [2-(8,9-dioxo-2.6-diazabicyclo[5.2.0]non-1(7)-en-2-yl)ethyl]phosphonic acid diethyl ester (37.6 g, 0.12 mole) in methylene chloride (50 mL). The reaction mixture is kept in a water bath at approximately 20° C. for 15 hr. The clear solution is concentrated under reduced pressure and the foamy residue is taken up in acetone (600 mL) with vigorous shaking to yield a thin suspension. Water (50 mL, 2.78 moles) is added to give a gummy precipitate which solidifies instantly. The suspension is shaken vigorously for 10 minutes, filtered, and washed with acetone to give a yellow solid compound. The solids are taken up in boiling water (450 mL) and the hot solution is filtered through a fluted filter paper to remove a small amount of insoluble material. The clear solution is cooled on ice to begin crystallization. The thick crystalline mass is diluted by the slow addition of acetone (800 mL), kept cold for one hour, filtered, and washed first with acetone and then with hexane to give a pale yellow solid (20.2 g). A second crop from the mother liquor (100% purity by LC) yields an additional (˜6.5 g) for a total yield of 87%. NMR (DMSO-d₆, 400 Mhz): 1.90 (m, 4H)), 3.25 (m, 2H), 3.36 (m, 2H), 3.84 (q, 4H), 8.45 (s, 1H). LC analysis: (Column: Nova Pak C18, 300×3.9 mm; Eluent: 20/80 MeOH/0.00r M Pic A; Flowrate: 1 mL/min; UV detectors at 210 nm). Analysis: Calc'd for C₉H₁₃N₂O₅P.1H₂O: C, 41.26; H, 5.08; N, 10.69%; Found: C, 41.17; H, 5.04; N, 10.42%; Karl-Fischer analysis: 0.55% H₂O; —FAB [M-H]⁻ m/z 259.

Scheme 2 depicts the preparation of, [2-(8,9-dioxo-2,6-diazabicyclo[5.2.0]non-1(7)-en-yl)alkyl]phosphonic by the following three-step protocol:

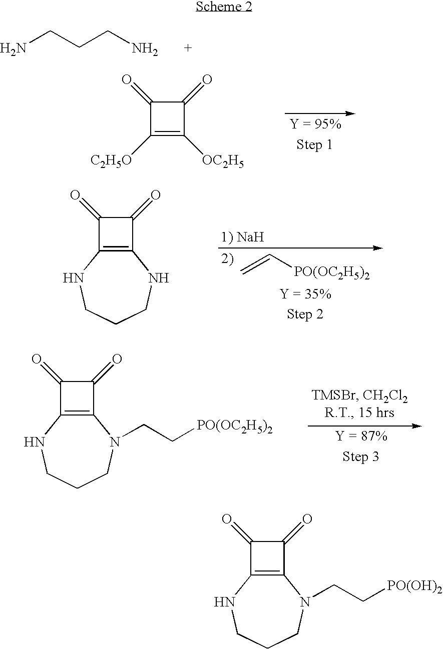

2,6-Diaza-bicyclo[5.2.0]non-1(7)-ene-8,9-dione

A solution of 3,4-diethoxy-3-cyclobutene-1,2-dione (6.8 g, 0.04 mole) in methanol (180 mL) and a solution of 1,3-diaminopropane (4.46 g, 0.06 mole) in MeOH (75 mL) are added dropwise in a parallel fashion over 10 minutes under dry nitrogen at ambient temperature to MeOH (100 mL) under vigorous stirring. The reaction mixture is stirred at ambient temperature overnight after which the precipitated product is filtered and washed with ice-cold MeOH (10 mL). The obtained faintly yellowish powder is dried under high vacuum, to yield ˜4.7 g (˜95%) of 2,6-Diaza-bicyclo[5.2.0]non-1(7)-ene-8,9-dione;(mp: 335° C.; MS (ES—): m/e 151.1 [M-H].

[2-(8,9-dioxo-2,6-diazabicyclo[5.2.0]non-1(7)-en-2-yl)ethyl]phosphonic acid diethyl ester

A suspension of 2,6-Diaza-bicyclo[5.2.0]non-1(7)-ene-8,9-dione (1.21 g, 0.08 mole) in N,N-dimethylformamide (75 mL) is treated under dry nitrogen and stirring with 60% sodium hydride in oil (0.328 g, 0.083 mole). After 30 minutes at room temperature, the reaction mixture is cooled to 0° C. and a solution of diethyl vinylphosphonate 97% (1.09 g, 0.08 mole) in N,N-dimethylformamide (20 mL) is added at once under vigorous stirring. The reaction is then stirred at room temperature overnight, concentrated under reduced pressure, and the residue is partitioned between 5% aqueous ammonium chloride solution (30 mL) and ethyl acetate (2×100 ml). The combined organic layers are washed with saturated sodium chloride (1×10 mL), dried over magnesium sulfate, filtered, and evaporated under reduced pressure to dryness. The residue is flash chromatographed on silica gel (60 g). Elution with 2% methanol in methylene chloride yields the title compound as a white solid (0.81 g, 35%) NMR (CDCl₃, 400 Mhz): 1.34 (t, 6H)), 2.06 (m, 2H), 2.20 (dt, 2H), 3.50 (m, 4H), 4.05 (m, 2H), 4.15 (m, 4H), 7.87 (br 1H).) MS (DEI) M⁺ m/z 316. LC analysis (column: Microsorb-MV C-18, 150×4.6 mm: Eluent 30/70 MeOH/0.01 M NH₄H₂PO₄ pH 4.7; Flow rate: 1 mL/min; UV detector at 210 nm; Analysis Calc'd for C₁₃H₂₁N₂O₅P: C, 49.36; H, 6.69; N, 8.85%; Found: C, 49.476; H, 6.74; N, 8.77%.

[2-(8,9-dioxo-2,6-diazabicyclo[5,2.0]non-1(7)-en-2-yl)ethyl]phosphonic acid

[2-(8,9-dioxo-2,6-diazabicyclo[5.2.0]non-1(7)-en-2-yl)ethyl]phosphonic acid is prepared using the same method as in Scheme 1.

Scheme 3 depicts the preparation of, [2-(8,9-dioxo-2,6-diazabicyclo[5.2.0]non-1(7)-en-yl)alkyl]phosphonic by the following three-step protocol:

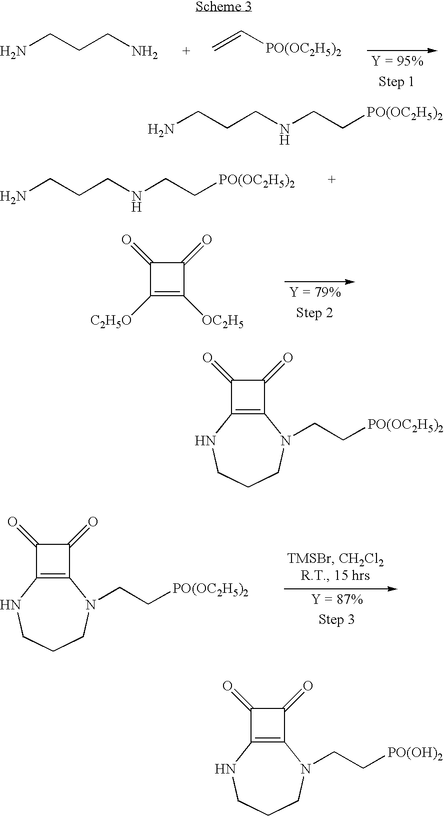

N-(3-aminopropyl)aminoethanephosphonic acid diethyl ester

To a 500 mL, three-necked flask, equipped with a magnetic stirrer and a nitrogen inlet, methanol (150 mL) and 1,3-diaminopropane (12.7 g, 0.152 mole, 5.0 equiv) is added (exothermic, 20° C. to 40° C.). The reaction mixture is stirred for 10 minutes and then diethyl vinylphosphonate 97% (5 g, 0.03 mole) in methanol (10 mL) is added in a stream. The mixture is stirred overnight at room temperature and the solvent is removed under reduced pressure, then the vacuum is increased to remove any unreacted 1,3-diaminopropane to give the product as a colorless oil (7.08 g, 98% yield). NMR (CDCl₃, 400 Mhz): 1.18 (t, 6H)), 1.47 (t, 2H), 1.80 (br, 3H), 1.83 (dt, 2H), 2.53 (t, 2H), 2.63 (dt, 2H), 2.76 (q, 2H), 3.95 (q, 4H).

[2-(8,9-dioxo-2,6-diazabicyclo[5.2.0]non-1(7)-en-2-yl)ethyl]phosphonic acid diethyl ester

To a 500 mL, three-necked flask, equipped with a magnetic stirrer and a nitrogen inlet, methanol (150 mL) is heated to 55-60° C. 3,4-diethoxy-3-cyclobutene-1,2-dione (1.04 g, 0.006 mole) is dissolved in methanol (50 mL) and the solution transferred to an addition funnel. Similarly, N-(3-aminopropyl)aminoethanephosphonic acid diethyl ester (1.46 g, 0.0061 mole) is dissolved in methanol (50 mL) and transferred to an addition funnel. The two solutions are concomitantly added dropwise into the preheated methanol over 5-6 hours. The mixture is stirred overnight at room temperature. The methanol is removed under reduced pressure and ethyl acetate (50 mL) is added to the residue. After cooling in an ice bath, the product is filtered and dried to yield (1.53 g, 79%). NMR (CDCl₃, 400 Mhz): 1.34 (t, 6H)), 2.06 (m, 2H), 2.20 (dt, 2H), 3.50 (m, 4H), 4.05 (m, 2H), 4.15 (m, 4H), 7.87 (br 1H).). MS (DEI) M⁺ m/z 316. LC analysis (column: Microsorb-MV C-18, 150×4.6 mm: Eluent 30/70 MeOH/0.01 M NH₄H₂PO₄ pH 4.7; Flow rate: 1 mL/min; UV detector at 210 nm.

[2-(8,9-dioxo-2,6-diazabicyclo[5.2.0]non-1(7)-en-2-yl)ethyl]phosphonic acid

[2-(8,9-dioxo-2,6-diazabicyclo[5.2.0]non-1(7)-en-2-yl)ethyl]phosphonic acid is prepared using the same method as in Scheme 1.

In other embodiments, the derivatives of the NMDA glutamate receptor antagonist [2-(8,9-dioxo-2,6-diazabicyclo[5.2.0]non-1-(7)-en-2-yl)alkyl]phosphonic acid depicted in formula (I), (II), and (III), as well as pharmaceutically acceptable salts thereof, may be synthesized by the methodology depicted in Scheme 4:

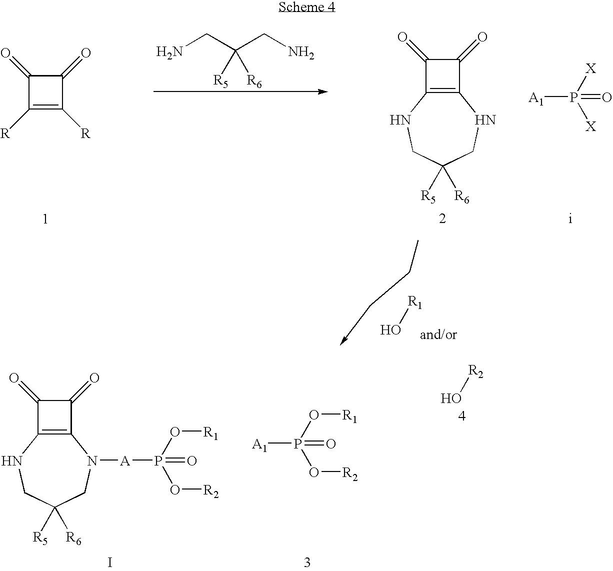

Reaction of a diaminoalkane with an dialkoxysquarate (1) in a suitable protic solvent, such as methanol, ethanol and the like, at a temperature ranging from about 0° C. to about 50° C., preferably at a temperature ranging from about 20° C. to about 30° C., provides the bicyclic intermediate of formula (2). By “suitable solvent” it is meant a solvent in which both the amine and the squarate are at least partially soluble and with which both are substantially non-reactive. Typically, the reaction time is about 10 hours to about 25 hours, and more preferably about 12 hours to about 18 hours.

In some embodiments, the diaminoalkane is diaminopropane (e.g., 1,3-diaminopropane). In other embodiments, R is C₁ to C₄ alkoxy. In still further embodiments, the dialkoxysquarate is diethoxysquarate wherein each R is —OEt. In some embodiments, R₅ and R₆ are both hydrogen. In further embodiments, R₅ and R₆ are, independently, hydrogen, alkyl, hydroxyl, alkoxy, or C₅ to C₇ aryl. Each of the alkyl, alkoxy, and C₅ to C₇ aryl may optionally be substituted as discussed above.

The anion of the bicyclic intermediate (2) can be formed by contacting (2) with a suitable base, such as a hydride or alkoxide, including, for example, sodium methoxide, potassium t-butoxide, sodium hydride or the like, in a suitable aprotic solvent, such as N,N-dimethylformamide or tetrahydrofuran. The anion is then treated with the phosphonate ester intermediate (3) wherein preferably A₁ is (CH₂)₂, but may be C₂-C₄ alkenyl or C₂-C₄ alkynyl, and preferably R₁ and R₂ are:

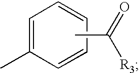

The mixture is stirred at ambient temperature from about 10 hours to about 25 hours, more typically from about 12 hours to about 18 hours. The desired compound of formula (I) is isolated from the reaction mixture using suitable purification techniques, such as flash chromatography or high-pressure liquid chromatography.

The phosphonate ester intermediate (3) can be prepared by alkylation of a compound of formula (4) with a phosphono dihalide (i) wherein X is a halide, A₁ is as defined above, and R₁ and R₂ are:

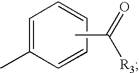

in a suitable aprotic solvent, such as dichloromethane or the like, at temperatures ranging from about 0° C. to about 30° C. In a preferred embodiment, A₁ is (CH₂)₂ and X is Cl. The reaction time is from about 10 hours to about 25 hours, and more typically from about 12 hours to about 16 hours. By “suitable solvent” it is meant a solvent in which both reagents are at least partially soluble and with which both reagents are substantially non-reactive. Preferably, an acid scavenger (to react with the acid halide by-product of the reaction), such as an organic amine, is optionally added to the reaction mixture in the reaction to form intermediate (3). The organic amine is typically a secondary amine or a tertiary amine such as triethylamine.

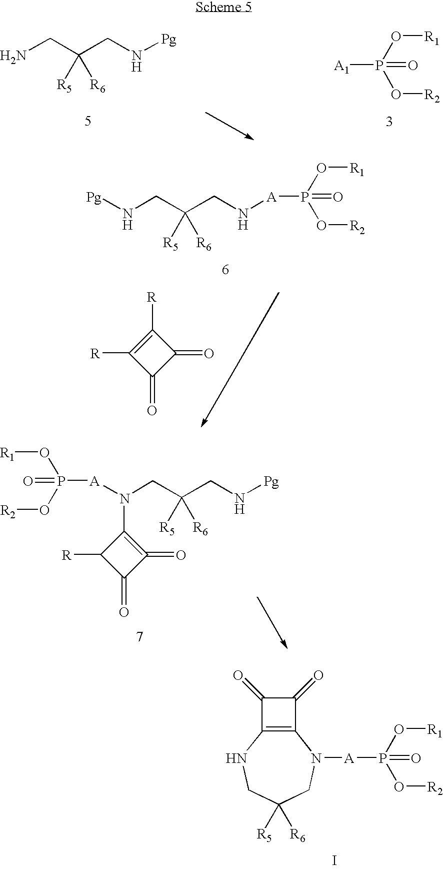

Alternatively, the compounds of formula (I), (II), (III), and pharmaceutically acceptable salts thereof, can be obtained as shown in Scheme 5 by adding the intermediate (3), one preparation of which is described above, to a mono-protected diaminoalkane (5) at ambient temperature and in a suitable aprotic solvent, such as tetrahydrofuran. The diaminoalkane may be mono-protected using a suitable protecting group (PG), such as t-butoxycarbonyl. The resulting disubstituted diaminoalkane derivative (6) is treated preferably at ambient temperature, with a dialkoxysquarate (1) in a suitable solvent, such as acetonitrile to provide the tri-substituted diaminoalkane derivative (7). The latter (7) is deprotected, for example, using trifluoroacetic acid in a suitable aprotic solvent, such as methylene chloride, after which cyclization is accomplished using, for example, an organic base, preferably a tertiary amine, such as triethylamine in a suitable solvent, such as acetonitrile. Those of skill in the art will readily recognize suitable protecting groups which may be used in this synthesis.

The syntheses of alternative exemplary [2-(8,9-dioxo-2,6-diazabicyclo[5.2.0]non-1-(7)-en-2-yl)alkyl]phosphonic acid derivatives including diethyl 2,2′-[({2-[8,9-dioxo-2,6-diazabicyclo[5.2.0]non-1(7)-en-2-yl]ethyl-}phosphoryl)bis(oxy)]dibenzoate; diethyl 4,4′-[({2-[8,9-dioxo-2,6-diazabicyclo[5.2.0]non-1(7)-en-2-yl]ethyl-}phosphoryl)bis(oxy)]dibenzoate; bis(4-acetylphenyl){2-[8,9-dioxo-2,6-diazabicyclo[5.2.0]non-1(7)-en-2-yl]e-thyl}phosphonate; bis(3-acetylphenyl){2-[8,9-dioxo-2,6-diazabicyclo[5.2.0]non-1(7)-en-2-yl]e-thyl}phosphonate; bis(2-acetylphenyl){2-[8,9-dioxo-2,6-diazabicyclo[5.2.0]non-1(7)-en-2-yl]e-thyl}phosphonate are described in U.S. Patent Publication No. 2006/0079679.

### Administration of NMDA Glutamate Receptor Antagonists to Achieve an Anesthetic-Sparing Effect

The NMDA glutamate receptor antagonist compositions of the present disclosure can be administered in any way known to those skilled in the art including, for example, by oral or parenteral administration, such as by intramuscular, intraperitoneal, epidural, intrathecal, intravenous, subcutaneous, intramucosal, such as sublingual or intranasal, vaginal, rectal or transdermal administration. In the embodiments disclosed herein, the NMDA glutamate receptor antagonist compositions are administered orally, intramucosally, intramuscularly, subcutaneously, or intravenously. The present disclosure is exemplified by parenteral administration of the anesthetic-sparing NMDA glutamate receptor antagonist [2-(8,9-dioxo-2,6-diazabicyclo[5.2.0]non-1-(7)-en-2-yl)alkyl]phosphonic acid prior to or after administration of the inhalant anesthetic isoflurane.

The compositions of the present disclosure, including compositions comprising the compounds of formula (I), (II), (III), and pharmaceutically acceptable salts thereof, are administered in an amount sufficient to achieve an anesthetic-sparing effect to a mammal in reducing the concentration (e.g., the minimum alveolar concentration or “MAC”) of anesthetics, especially inhalant anesthetics, required to maintain anesthesia (i.e. achieving an “anesthetic-sparing” effect). As used herein “an anesthetic-sparing amount” is at least the minimal amount of the compound or a pharmaceutically acceptable salt form thereof, which is required to achieve an anesthetic-sparing effect for the anesthetic to be administered. The anesthetic sparing amount will depend on such variables as the particular compound used, the route of administration, the nature of the anesthetic, and the particular subject being treated.

To determine the anesthetic-sparing amount of the compound to be administered, the veterinarian or physician may, for example, evaluate the effects of a given compound of formula (I), (II), (III), and pharmaceutically acceptable salts thereof, in the subject by incrementally increasing the dosage until the desired anesthetic-sparing effect is achieved. The continuing dose regimen may then be modified to achieve the desired result. For example, in the case of an intravenous (IV) dosage, the compounds of the present disclosure may be incrementally increased in a subject over an approximate range of 5 mg/kg to 20 mg/kg until the desired anesthetic-sparing effect is achieved. Further doses could be administered as needed, although the examples provided herein demonstrate undiminished efficacy over a period of up to 5 hours after a single IV administration. Similar techniques may be followed by determining the effective dose range for other administration routes, such as by subcutaneous, intramuscular, or oral based on bioavailability and/or efficacy data.

In another embodiment, the compositions of the present disclosure, including compositions comprising the compounds of formula (I), (II), (III), and pharmaceutically acceptable salts thereof, may be administered to a mammal with one or more of the various other pharmaceutical active agents used in the perioperative setting. Examples of such pharmaceutical active agents include analgesic agents, muscle-relaxing agents, hypnotic/dissociative agents, anesthetics, or combinations thereof. These agents could be members of such pharmaceutical classes as benzodiazepines (e.g., zolazepam and valium), opioids (e.g., morphine, butorphanol, and fentanyl), α-2 adrenergic agonists (e.g., medetomidine and xylazine), a non-steroidal anti-inflammatory drug (NSAID) (e.g., etodolac, carprofen, deracoxib, firocoxib, tepoxalin, and meloxicam), corticosteroids (e.g., cortisol), barbiturates (e.g., thiopental and phenobarbital), channel-blocking NMDA antagonists (e.g., ketamine and tiletamine), anesthetics including inhalant (e.g., sevoflurane, halothane) and injectable (e.g., etomidate, propofol and alfaxan) classes. This is not intended to be a comprehensive listing of pharmaceutically active agents that may potentially be administered in combination with perzinfotel. A more complete listing of pharmaceutically active agents can be found in the Physicians' Desk Reference, 55^{th} Edition, 2001, published by Medical Economics Co., Inc., Montvale, N.J. and in the Compendium of Veterinary Products (CVP), 10^{th} Edition, 2007, published by North American Compendiums; Inc., Port Huron, Mich. Each of these agents may be administered according to the therapeutically effective dosages and regimens known in the art, such as those described for the products in the Physicians' Desk Reference, 55th Edition, 2001, published by Medical Economics Co., Inc., Montvale, N.J.

The one or more other pharmaceutically active agents may be administered in a therapeutically effective amount simultaneously (such as individually at the same time, or together in a pharmaceutical composition), and/or successively with one or more composition of the present disclosure, including compositions comprising the compounds of formula (I), (II), (III), and pharmaceutically acceptable salts thereof.

The method of administration of the other pharmaceutically active agent may be the same or different from the route of administration used for the compositions of the present disclosure. For example, the other pharmaceutically active agents may be administered by oral or parenteral administration such as, for example, by intramuscular, intraperitoneal, epidural, intrathecal, intravenous, intramucosal (e.g., intranasal or sublingual), subcutaneous, or transdermal administration. The preferred administration route will depend upon the particular pharmaceutically active agent chosen and its recommended administration route(s) known to those skilled in the art.

One skilled in the art will recognize that the dosage of these other pharmaceutical active agents administered to the mammal will depend on the particular agent in question and the desired administration route. Accordingly, the other pharmaceutically active agent(s) may be dosed and administered according to those practices known to those skilled in the art, such as those disclosed in references, such as the Physicians' Desk Reference, 55th Edition, 2001, published by Medical Economics Co., Inc., Montvale, N.J.

Within certain embodiments of the present invention, a composition comprising an anesthetic-sparing compound of formula (I), (II), and/or (III) may be administered with at least one opioid analgesic in accordance with the methods previously described herein. When administered with at least one opioid analgesic, such as morphine or fentanyl (as disclosed, for example, in Example 2), compositions comprising an anesthetic-sparing compound of formula (I), (II), and/or (III) may have such beneficial effects as synergistically decreasing pain perception and/or anesthetic-sparing effect.

The anesthetic-sparing compositions of the present disclosure, including compositions comprising compounds of formula (I), (II), (III), and pharmaceutically acceptable salts thereof, may be administered neat (i.e. as is) or in a pharmaceutical composition containing at least one pharmaceutically acceptable carrier. Thus, the present invention also provides pharmaceutical compositions containing a pharmaceutically effective amount of at least one compound of formula (I), (II), (III), and pharmaceutically acceptable salts thereof, and at least one pharmaceutically acceptable carrier. Preferred compounds to be present in the pharmaceutical compositions of the present invention include those compounds of formula (I), (II), (III), and pharmaceutically acceptable salts thereof previously described as being preferred. Pharmaceutically acceptable carriers are those that are compatible with the other ingredients in the formulation and biologically acceptable.

Pharmaceutical compositions useful as anesthetic-sparing compositions may be in any form known to those skilled in the art, such as in liquid or solid form. The proportion of ingredients will depend on such factors as the solubility and chemical nature of the compound of formula (I), (II), (III), and pharmaceutically acceptable salts thereof, and the chosen route of administration. Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985).

Pharmaceutical compositions, in addition to containing an anesthetic-sparing amount of one or more of the compounds disclosed herein and a pharmaceutically acceptable carrier may include one or more other ingredients known to those skilled in the art for formulating pharmaceutical compositions.

Solid pharmaceutical compositions may contain one or more anesthetic-sparing compounds of the present disclosure and one or more solid carriers, and optionally one or more other additives, such as flavoring agents, lubricants, solubilizers, suspending agents, fillers, glidants, compression aids, binders or tablet-disintegrating agents or an encapsulating material. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes or ion exchange resins, or combinations thereof. In powder pharmaceutical compositions, the carrier may be a finely divided solid that is in admixture with the finely divided active ingredient. In tablets, the active ingredient may be mixed with a carrier having the necessary compression properties in suitable proportions, and optionally, other additives, and compacted into the desired shape and size. Solid pharmaceutical compositions, such as powders and tablets, preferably contain up to 99% of the active ingredient.

Liquid pharmaceutical compositions may contain one or more anesthetic-sparing compounds of the present disclosure and one or more liquid carrier(s) to form for example solutions, suspensions, emulsions, syrups, elixirs, or pressurized compositions. Pharmaceutically acceptable liquid carriers include for example water, organic solvent, pharmaceutically acceptable oils or fat, or combinations thereof. The liquid carrier can contain other suitable pharmaceutical additives, such as solubilizers, emulsifiers, buffers, preservatives, sweeteners, flavoring agents, suspending agents, thickening agents, colors, viscosity regulators, stabilizers or osmo-regulators, or combinations thereof.

Examples of liquid carriers suitable for oral or parenteral administration include water (preferably containing additives, such as cellulose derivatives, such as sodium carboxymethyl cellulose), alcohols or their derivatives (including monohydric alcohols or polyhydric alcohols, such as glycols) or oils (e.g., fractionated coconut oil and arachis oil). For parenteral administration, the carrier can also be an oily ester, such as ethyl oleate and isopropyl myristate. The liquid carrier for pressurized compositions can be halogenated hydrocarbons or other pharmaceutically acceptable propellant.

Liquid pharmaceutical compositions that are sterile solutions or suspensions can be administered parenterally for example by intramuscular, intraperitoneal, epidural, intrathecal, intravenous, or subcutaneous injection. Pharmaceutical compositions for oral or transmucosal administration may be either in liquid or solid composition form.

Anesthetic-sparing compositions, including pharmaceutical compositions, may be in unit dosage form, such as tablets or capsules. In such form, the anesthetic-sparing composition is sub-divided in unit dose containing appropriate quantities of the active ingredient including, for example, a compound of formula (I), (II), and/or (III), and/or pharmaceutically acceptable salts thereof. The unit dosage forms can be packaged compositions, for example packeted powders, vials, ampoules, pre-filled syringes, or sachets containing liquids. The unit dosage form can be, for example, a capsule or tablet itself, or it can be the appropriate number of any such compositions in package form.

Thus, the present disclosure provides pharmaceutical compositions in unit dosage form that contain a therapeutically effective unit dosage of at least one anesthetic-sparing compound of the present invention. As one skilled in the art will recognize, the preferred unit dosage will depend on for example the method of administration and the condition being treated. For example, a unit dosage may range from about 1 mg of anesthetic-sparing compound/kg of body-mass to about 1 g of anesthetic-sparing compound/kg of body-mass; from about 2 mg of anesthetic-sparing compound/kg of body mass to about 100 mg of anesthetic-sparing compound/kg of body-mass; or from about 5 mg of anesthetic-sparing compound/kg of body-mass to about 20 mg of anesthetic-sparing compound/kg of body-mass.

The present invention also provides a therapeutic package for dispensing the compound of the present invention, including compounds of formula (I), (II), (III), and pharmaceutically acceptable salts thereof, to a mammal being treated. The therapeutic package may contain one or more unit dosages of the anesthetic-sparing compound of the present invention and a container containing the one or more unit dosages and labeling directing the use of the package for achieving an anesthetic-sparing effect in a mammal.

### Anesthetic Agents

Typically, the anesthetics employed in combination with the NMDA glutamate receptor antagonists presented herein are general anesthetics. General anesthetics are anesthetic drugs that bring about a reversible loss of consciousness. A general anesthetic, when properly administered, will cause a progressive depression of the central nervous system so that the patient loses consciousness. As used herein, the phrase “general anesthesia” refers to the induction of a balanced state of unconsciousness, accompanied by the absence of pain sensation and the relaxation of skeletal muscle over the entire body. It is induced through the administration of anesthetic drugs and is used during major surgery and other invasive surgical procedures.

The objectives of general anesthesia administered prior to a surgical operation, may include: a) blocking the patient's movements and relaxing the patient's muscles to prevent involuntary reflex muscle movements which may interfere with the operation (i.e. produce muscle relaxation); b) preventing the patient from being aware (i.e. loss of consciousness, or sedation) during the operation; c) preventing the patient feeling pain (i.e. loss of sensation, or analgesia) during the operation; and d) preventing the patient from remembering intra-operative events or discussions (i.e. amnesia). The anesthesia should not lower blood pressure to a dangerous extent (e.g., below about 60 mm Hg or about 50 mm Hg for mean arterial pressure (MAP)). In order to monitor the “anesthetic depth” or “plane of anesthesia” of the patient, a skilled anesthesiologist monitors selected physiological parameters that indicate the vital signals of the patient (e.g., breathing, blood pressure, etc.) and bispectral index (BIS), a numerical score derived from EEG data which ranges from between about 30 and about 65 at the levels of unconsciousness achieved in surgical settings) to about 100 (fully conscious), to determine if more or less anesthetic is required.

Within certain embodiments, general anesthetics may be inhalational or intravenous anesthetics. Inhalational anesthetics, which are gases or vapors possessing anesthetic qualities, include the volatile anesthetics halothane, isoflurane, sevoflurane, and desflurane and the gases ethylene, cyclopropane, ether, chloroform, nitrous oxide, and xenon. Inhalation anesthetics or volatile anesthetics are compounds that enter the body through the lungs and are carried by the blood to body tissues. Inhalation anesthetics are typically used in combination with nonvolatile intravenous anesthetics that are administered by injection or intravenous infusion. Intravenous general anesthetics include ketamine, tiletamine, thiopental, methohexital, etomidate, and propofol.

The anesthetic-sparing effects of perzinfotel are exemplified herein by combination with the anesthetic isoflurane. It will be understood that a wide variety of anesthetic compounds may be satisfactorily employed in the anesthetic sparing methods disclosed herein. For example, the present disclosure contemplates the use of alternative fluoroether compounds that are, in addition to isoflurane, commonly employed as anesthetic agents. Examples of suitable fluoroether compounds used as anesthetic agents include sevoflurane (fluoromethyl-2,2,2-trifluoro-1-(trifluoromethyl)ethyl ether); enflurane ((±-)-2-chloro-1,1,2-trifluoroethyl difluoromethyl ether); isoflurane (1chloro-2,2,2-trifluoroethyl difluoromethyl ether); methoxyflurane (2,2-dichloro-1,1-difluoroethyl methyl ether); and desflurane ((±-)-2-difluoromethyl 1,2,2,2-tetrafluoroethyl ether). Other anesthetics, such as halothane, may also be employed.

The following patents that describe methods and apparatus for monitoring and/or controlling the provision of anesthetic to patients are hereby incorporated by reference in their entirety: U.S. Pat. No. 6,315,736 to Tsutsumi et al.; U.S. Pat. No. 6,317,627 to Ennen et al.; U.S. Pat. No. 6,016,444 to John; U.S. Pat. No. 5,699,808 to John; U.S. Pat. No. 5,775,330 to Kangas et al.; U.S. Pat. No. 4,557,270 to John; U.S. Pat. No. 5,010,891 to Chamoun; and U.S. Pat. No. 4,869,264 to Silberstein.

### EXAMPLES

The present disclosure will be better understood by reference to the following non-limiting examples:

### Example 1

### The NMDA Glutamate Receptor Antagonist Perzinfotel as an Anesthetic-Sparing Agent

This Example demonstrates that the NMDA glutamate receptor antagonist perzinfotel is effective in reducing the Minimum alveolar concentration (MAC) of isoflurane required to maintain anesthesia in dogs.

MACs for isoflurane were determined for six dogs before and after administering IV bolus doses of perzinfotel, formulated as a sterile aqueous solution containing 50 mg/ml of perzinfotel, 8.3 mg/ml of sodium hydroxide (NaOH), and 0.4 mg/ml of ethylenediamine tetraacetic acid (EDTA). Anesthesia was defined as unconsciousness and non-responsiveness to a severely noxious stimulus (electric shock).

Table 1 presents the effects of the NMDA glutamate receptor antagonist perzinfotel on Minimum Alveolar Concentration (MAC) of Isoflurane required to maintain anesthesia. MAC values are presented as %s of isoflurane in exhaled (end-tidal) gases. “BASELINE” MAC values were established first and used to set each dog's initial isoflurane dose in later determinations. To evaluate the effects of perzinfotel, control MACs were first determined approximately 1 hour after administering IV saline, followed by IV administration of perzinfotel 3-5 min. after determining control MAC, and two more MAC determinations approximately 2 hours (“1st) and 5 hours (“2nd”) after administration of perzinfotel.

The average MAC values following the administration of 5, 10 and 20 mg/kg IV perzinfotel were 1.01, 0.93, and 0.71, respectively (Table 1). These MAC values were significantly lower than control or baseline MAC values (averaging about 1.3%) and were significantly different from each other. These data demonstrate that the NMDA glutamate antagonist perzinfotel is effective in reducing the MAC of isoflurane required to maintain anesthesia in dogs.

TABLE 1
Effects of the NMDA Glutamate Receptor Antagonist
Perzinfotel on Minimum Alveolar Concentration (MAC)
of Isoflurance Required to Maintain Anesthesia¹

| MEAN MINIMUM ALVEOLAR | | | | |
|---|---|---|---|---|
| CONCENTRATION (MAC %) OF ISOFLURANE | | | | |
| REQUIRED TO MAINTAIN ANESTHESIA (NO | | | | |
| RESPONSE TO NOXIOUS STIMULUS) | | | | |
| TREATMENTS | | | | % DECREASE |
| IV SALINE | | | | MAC (Relative to |
| (Control) | 1^{st} | 2^{nd} | AVERAGE | Saline) |
| | BASELINE (no other treatments) | | | |
| NA² | 1.33 | 1.33 | 1.33 | NA² |
| | IV 5 MG/KG PERZINFOTEL | | | |
| 1.33 | 1.03 | 0.99 | 1.01 | 22.73 |
| | IV 10 MG/KG PERZINFOTEL | | | |
| 1.32 | 0.93 | 0.93 | 0.93 | 28.62 |
| | IV 20 MG/KG PERZINFOTEL | | | |
| 1.32 | 0.72 | 0.70 | 0.71 | 45.33 |

¹n = 6 dogs
²NA = Not Applicable

Bispectral index (BIS), a measure of consciousness/hypnosis, was calculated from electroencephalographic data collected concurrently with the MAC determinations. BIS values after administration of perzinfotel were unchanged or increased relative to the baseline and saline controls. This indicates that the effects of perzinfotel on MAC were probably mediated through analgesic rather than anesthetic mechansism(s) since BIS correlates with level of consciousness and was not decreased, as would be expected with supplemental anesthesia.

Table 2 presents the effects of perzinfotel on bispectral index. Bispectral index was calculated from electroencephalogram (EEG) data collected concurrently with the MAC determinations shown in Table 1. BIS values were calculated from EEG readings taken immediately prior to noxious stimulation.

TABLE 2
Effects of Perzinfotel on Bispectral Index
MEAN BISPECTRAL INDEX (BIS)
TREATMENTS

| | | |
|---|---|---|
| IV Saline | | |
| (Control) | 1^{st} | 2^{nd} |

BASELINE (No Other Treatments)

| | | |
|---|---|---|
| NA¹ | 61 | 63 |

IV 5 MG/KG PERZINFOTEL

| | | |
|---|---|---|
| 69 | 70 | 68 |

IV 10 MG/KG PERZINFOTEL

| | | |
|---|---|---|
| 59 | 69 | 79 |

IV 20 MG/KG PERZINFOTEL

| | | |
|---|---|---|
| 63 | 81 | 78 |

¹Not Applicable

Hemodynamic and respiratory parameters were also collected concurrently with MAC determinations. These included body temperature, respiratory rate, median arterial blood pressure (MAP), heart rate, percent saturation of hemoglobin with oxygen, (SpO₂), systolic arterial blood pressure (SAP), diastolic arterial blood pressure (DAP), end-tidal [exhaled] oxygen concentration (ETO₂), and end-tidal [exhaled] carbon dioxide concentration (ETCO₂). These results, shown in Table 3, indicate that perzinfotel acted to reduce isoflurane-induced depression of hemodynamics. For example, at 10 and 20 mg/kg perzinfotel, all blood pressure parameters (MAP, SAP, and DAP) were significantly different from control levels with isoflurane alone. The MAP results in particular show that isoflurane depressed blood pressure below normal conscious levels, and addition of perzinfotel restored blood pressure significantly toward the conscious range. The same pattern, was observed for heat rate.

Table 3 presents a summary of hemodynamic and respiratory parameters following administration of perzinfotel (EAA-090) and isoflurane. Hemodynamic and respiratory parameters were measured concurrently with the MAC determinations shown in Table 1, except for conscious dog data.

TABLE 3
Summary of Mean Hemodynamic and Respiratory Parameters following Administration
of Perzinfotel and Isoflurane

| | | DOGS ANESTHETIZED WITH ISOFLURANE | | | | | |
|---|---|---|---|---|---|---|---|
| | | BASELINE | | | | | |
| | CONSCIOUS | (no other treatment) | | IV SALINE | IV PERZINFOTEL | | |
| PARAMETER | DOGS | 1^{st} | 2^{nd} | (Control) | DOSE | 1^{st} | 2^{nd} |
| Heart Rate | 132 | 101 | 115 | 111 | 5 mg/kg | 129 | 113 |
| (beats/min) | | | | 104 | 10 mg/kg | 132 | 140 |
| | | | | 102 | 20 mg/kg | 134 | 141 |
| MAP | 134 | 71 | 79 | 75 | 5 mg/kg | 90 | 88 |
| (mm Hg) | | | | 80 | 10 mg/kg | 98 | 96 |
| | | | | 73 | 20 mg/kg | 105 | 106 |
| SAP | data not | 95 | 105 | 102 | 5 mg/kg | 116 | 114 |
| (mm Hg) | available | | | 107 | 10 mg/kg | 126 | 124 |
| | | | | 97 | 20 mg/kg | 138 | 139 |
| DAP | data not | 58 | 63 | 60 | 5 mg/kg | 73 | 69 |
| (mm Hg) | available | | | 66 | 10 mg/kg | 82 | 80 |
| | | | | 57 | 20 mg/kg | 86 | 85 |
| Respiratory | data not | 12 | 29 | 12 | 5 mg/kg | 24 | 27 |
| Rate | available | | | 12 | 10 mg/kg | 26 | 34 |
| (breaths/min) | | | | 12 | 20 mg/kg | 29 | 16 |
| Sp0₂ | data not | 99 | 99 | 99 | 5 mg/kg | 99 | 100 |
| (%) | available | | | 99 | 10 mg/kg | 99 | 100 |
| | | | | 100 | 20 mg/kg | 99 | 99 |
| Body | 38.5 | 37.9 | 37.9 | 37.8 | 5 mg/kg | 37.8 | 37.8 |
| Temperature | | | | 37.9 | 10 mg/kg | 37.9 | 38.0 |
| (° C.) | | | | 37.9 | 20 mg/kg | 37.9 | 37.9 |
| ETO₂ | data not | 93 | 93 | 95 | 5 mg/kg | 94 | 94 |
| (mm Hg) | available | | | 94 | 10 mg/kg | 94 | 95 |
| | | | | 94 | 20 mg/kg | 95 | 94 |
| ETCO₂ | data not | 42 | 37 | 40 | 5 mg/kg | 32 | 30 |
| (mm Hg) | available | | | 38 | 10 mg/kg | 37 | 32 |
| | | | | 39 | 20 mg/kg | 31 | 30 |

### Example 2

### Cooperative Interactions between NMDA Glutamate Receptor Antagonist Perzinfotel and an Opioid Agonist, Fentanyl

This Example demonstrates the cooperative interaction between the NMDA glutamate receptor antagonist perzinfotel and the opioid agonist fentanyl.

It is highly desirable that novel drugs introduced for perioperative use (e.g., anesthetic-sparing agents) be compatible with existing anesthetic adjuvants. For this reason, the anesthetic-sparing effects (relative to isoflurane alone) were determined in dogs for three treatments: 1. Perzinfotel (20 mg/kg IV bolus); 2. Fentanyl (5 μg/kg IV bolus followed by 0.15 μg/kg/min. IV infusion); 3. Combination of fentanyl and perzinfotel (dosed as in 1. and 2.). Fentanyl was chosen for this example because it is a commonly used analgesic compound for surgical procedures and because U.S. Pat. No. 7,098,200 discloses especially favorable interactions between perzinfotel and opioid analgesics.

The comparative effects of perzinfotel, fentanyl, and fentanyl:perzinfotel (combination) on Minimum Alveolar Concentration (MAC) of isoflurane are presented in Table 4, which demonstrate that the anesthetic-sparing effects of fentanyl and perzinfotel are highly complementary. The mean anesthetic-sparing effect of the fentanyl:perzinfotel combination, 66%, was approximately the sum of the separate effects of perzinfotel and fentanyl (39% and 34% respectively). Cardiopulmonary function of dogs anesthetized with isoflurane and administered the fentanyl:perzinfotel combination was not reduced below that of dogs anesthetized with isoflurane and administered fentanyl alone. The anesthetic-sparing effect of the fentanyl:perzinfotel combination is greater than can be achieved safely by fentanyl alone. For example, higher doses of fentanyl can produce thoracic rigidity (in addition to the typical opioid-induced respiratory suppression), bradyarythmia, hypothermia, and loss of sphincter tone. Basic methods were similar to those described in Table 1 (note, however, that a different group of 6 dogs was used for these experiments). “BASELINE” MAC values were determined approximately 1.4 hours (“1st) and 5.5 hours (2nd) after starting isoflurane (no other treatment). Control MACs were determined approximately 1.5 hours after administering IV saline. MACs influenced by fentanyl were determined approximately 1.5 hours after beginning fentanyl administration (initial IV bolus followed by constant rate IV infusion). Perzinfotel (IV bolus) was administered 3-5 min. after determination of fentanyl-influenced MACs (with fentanyl infusions continued until the end of the experiment). MACs influenced by the fentanyl:perzinfotel combination were determined approximately 1 hour (“1st”) and 3 hours (“2nd”) after administration of Perzinfotel.

TABLE 4
Comparative Effects of Perzinfotel, Fentanyl, and Fentanyl:Perzinfotel (combination)
on Minimum Alveolar Concentration (MAC) of Isoflurane
MEAN MINIMUM ALVEOLAR CONCENTRATION (MAC) OF ISOFLURANE REQUIRED TO MAINTAIN ANESTHESIA
(NO RESPONSE TO NOXIOUS STIMULUS)
TREATMENTS

| | | | | | | | | FENTANYL AND FENTANYL:PERZINFOTEL | | | | | | |
|---|---|---|---|---|---|---|---|---|---|---|---|---|---|---|
| | | | | | | | | | FENTANYL = | | | | | |
| | | | | | | | | | 5 μg/kg IV bolus | | FENTANYL:PERZINFOTEL = | | | |
| | | | | | | | | | followed by 0.15 | | 20 mg/kg IV Perzinfotel | | | |
| | | | PERZINFOTEL | | | | | | μg/kg/min | | (and continuing with IV | | | |
| | | | | 20 MG/KG IV PERZINFOTEL | | | | | IV infusion. | | infusion of fentanyl). | | | |
| BASELINE | | | | | | | % | | | % | | | | % |
| (no other | | | IV | | | | Decrease | IV | | Decrease | | | | Decrease |
| treatments) | | | SALINE | | | | (Relative | SALINE | Fentanyl | (Relative | | | | (Relative |
| 1^{st} | 2^{nd} | Avg. | (Control) | 1^{st} | 2^{nd} | Avg. | to Saline) | (Control) | (1 only) | to Saline) | 1^{st} | 2^{nd} | Avg. | to Saline) |
| 1.38 | 1.44 | 1.41 | 1.42 | 0.87 | 0.87 | 0.87 | 38.7 | 1.42 | 0.93 | 34.1 | 0.50 | 0.47 | 0.48 | 65.9 |

In summary, administration of IV bolus doses of perzinfotel of 5, 10, and 20 mg/kg produced dose-dependent, anesthetic-sparing reductions in MAC for isoflurane. The effects of a single dose of perzinfotel were sustained for at least 5 hours (longest interval between dosing and second MAC determination). The MAC reductions probably resulted from analgesic mechanisms (as opposed to anesthetic) since concurrent BIS values were unchanged or increased (toward increased consciousness). From other concurrent measurements, body temperatures were unchanged, respiratory rates were unchanged or increased, all blood pressure indices were increased, and heart rates were unchanged or increased (all results relative to vehicle controls in isoflurane anesthetized dogs). Even greater MAC reductions were produced by combining perzinfotel with the opioid analgesic fentanyl. Thus, perzinfotel is highly complementary to at least one of the drugs commonly used along with inhalant anesthetics without sacrificing cardiopulmonary safety.

### Example 3

The study was conducted using a six-treatment Latin squared crossover design. Six dogs were assigned to each treatment. Each dog received all doses/routes of perzinfotel throughout the duration of the study; however, only a single treatment was administered at a given time. The treatments are displayed in Table 5.

A baseline/control MAC of isoflurane (MACₒ) was determined following pretreatment with the control article (saline). At least one week (7 days) later, the MAC was re-determined after administration of one of the treatments in Table 5.

TABLE 5
Treatment overview

| Treatment | Dosing Rate |
|---|---|
| A | 20 mg/kg Perzinfotel IV |
| B | 20 mg/kg Perzinfotel SQ |
| C | 20 mg/kg Perzinfotel IM |
| D | 10 mg/kg Perzinfotel IM |
| E | 30 mg/kg Perzinfotel IM |
| F | 20 mg/kg Perzinfotel IM + 0.2 mg/kg |
| | butorphanol IM |

Following treatment (Table 5), general anesthesia was established and MAC was re-determined twice: approximately 15 min after anesthesia onset (MAC1), and two hours later (MAC2). This process was repeated for the remaining treatments at an interval of approximately 7 days.

In addition to MAC values, arterial blood pressure, electrocardiogram (ECG), respiratory rate, oxygen saturation with hemoglobin (SpO₂), end tidal gases (oxygen, carbon dioxide, and isoflurane) and BIS values were measured.

Under control conditions (i.e., administration of saline), the MAC of isoflurane needed to prevent gross purposeful movement in response to a noxious (electrical) stimulus, were 1.13 and 1.20 when determined approximately 15min after anesthesia onset and 2hrs later, respectively. As displayed in Table 6, perzinfotel substantially decreased the isoflurane MAC at all doses and by all routes (IV, IM, SC) of administration.

All doses and routes of administration of perzinfotel increased BIS; perzinfotel also decreased the amount of cardiopulmonary depression produced by the isoflurane anesthesia. The co-administration of butorphanol, 0.2 mg/kg IM, and perzinfotel, 20 mg/kg IM, produced the largest decrease in isoflurane MAC. This effect was sustained for the duration of the experiment.

The data in Examples 1-3 demonstrate that the NMDA glutamate receptor antagonist perzinfotel is effective in achieving an anesthetic-sparing effect for the anesthetic isoflurane. Thus, when administered during a surgical procedure, perzinfotel allows effective anesthesia to be produced by reduced amounts of an anesthetic compound. These effects are most likely mediated through analgesic mechanism(s) in the central nervous system. Effective anesthesia with less risk of complications from suppression of central homeostatic mechanisms (e.g., improved cardiopulmonary function) represents a substantial benefit to surgical patients.

TABLE 6
Mean Minimum alveolar concentration (MAC), during isoflurane
anesthesia following control (CTRL) and PERZINFOTEL
pre-treatment; MAC values determined twice, first immediately
after induction of anesthesia (1^{st} MAC) and 2 hours later
(2^{nd} MAC); n = 6 dogs.

| | | Treatment^{1,2} | | | | | | | |
|---|---|---|---|---|---|---|---|---|---|
| | | CTRL | A | B | C | D | E | F | G |
| MAC | 1^{st} | 1.13 | 0.65 | 0.75 | 0.70 | 0.75 | 0.63 | 0.43 | 1.12 |
| (%) | | — | −43% | −34% | −38% | −34% | −44% | −61% | −1% |
| | 2^{nd} | 1.20 | 0.83 | 0.78 | 0.75 | 0.80 | 0.65 | 0.53 | 0.97 |
| | | — | −31% | −35% | −38% | −33% | −46% | −56% | −19% |

¹CTRL: Control (Saline) treatment at the beginning of the experiment; PERZINFOTEL: A (20 mg/Kg IV), B (20 mg/Kg SQ), C (20 mg/Kg IM), D (10 mg/Kg IM), E (30 mg/Kg IM), and F (20 mg/Kg IM + BUTORPHANOL 0.2 mg/Kg IM).
²G: Control (Saline) treatment for 1^{st} MAC, and BUTORPHANOL (0.2 mg/Kg IM) for 2^{nd} MAC.

What is claimed is:

1. A method for achieving an anesthetic-sparing effect in a subject, said method comprising, administering to said subject an NMDA glutamate receptor antagonist and a general anesthetic; wherein an anesthetic-sparing effect is achieved in the subject.

2. The method of claim 1, wherein said NMDA glutamate receptor antagonist is a compound of formula (I) or a pharmaceutically acceptable salt or tautomer thereof: 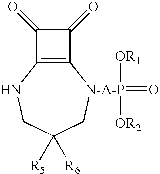
wherein A is alkylenyl of 1 to 4 carbon atoms;
R₁ and R₂ are, independently, hydrogen or phenyl optionally substituted with 1 to 2 substituents, independently, selected from the group consisting of —C(O)R₃, halogen, cyano, nitro, hydroxyl, C₁-C₆ alkyl, and C₁-C₆ alkoxy;
R₃ is, independently, hydrogen, —OR₄, alkyl, aryl, or heteroaryl;
R₄ is hydrogen, alkyl, aryl, or heteroaryl;
R₅ and R₆ are, independently, hydrogen, alkyl, hydroxyl, alkoxy, or phenyl;
wherein any R₃ to R₆ group having an aryl or heteroaryl moiety can optionally be substituted on the aryl or heteroaryl moiety with 1 to about 5 substituents, independently, selected from the group consisting of halogen, cyano, nitro, hydroxyl, C₁-C₆ alkyl, and C₁-C₆ alkoxy.

3. The method of claim 1, wherein said NMDA glutamate receptor antagonist is [2-(8,9-dioxo-2,6-diazabicyclo[5.2.0]non-1-(7)-en-2-yl)ethyl]phosphonic acid or a tautomer or pharmaceutically acceptable salt thereof.

4. The method of claim 1, wherein said NMDA glutamate receptor antagonist is diethyl 3,3′-[({2-[8,9-dioxo-2,6-diazabicyclo[5.2.0]non-1(7)-en-2-yl]ethyl}phosphoryl)bis(oxy)] dibenzoate or a tautomer or pharmaceutically acceptable salt thereof.

5. The method of claim 1, wherein said general anesthetic is administered via inhalation or intravenously.

6. The method of claim 1, wherein said NMDA glutamate receptor antagonist is administered parenterally.

7. The method of claim 1, further comprising administering an additional anesthetic agent.

8. The method of claim 1, wherein said general anesthetic is selected from the group consisting of halothane, isoflurane, sevoflurane, desflurane, ethylene, cyclopropane, ether, chloroform, nitrous oxide, and xenon.

9. The method of claim 8, wherein said general anesthetic is isoflurane.

10. The method of claim 1, wherein said general anesthetic is selected from the group consisting of ketamine, thiopental, methohexital, etomidate, propofol, flumazenil, retamine, remifentanyl, midazolam, pentothal, and evipal procaine.

11. The method of claim 7, wherein the general anesthetic is isoflurane and the additional anesthetic agent is propofol.

12. The method of claim 1, further comprising the step of administering to said subject one or more pharmaceutically active agent selected from the group consisting of an analgesic agent, a muscle-relaxing agent, and a hypnotic/dissociative agent.

13. The method of claim 1, further comprising the step of administering to said subject one or more pharmaceutically active agent selected from the group consisting of a benzodiazepine, an opioid, an α-2 adrenergic agonist, a non-steroidal anti-inflammatory drug (NSAID), a corticosteroid, a barbiturate, a non-barbiturate hypnotic a dissociative, a channel-blocking NMDA antagonist, and an injectable.

14. The method of 13, wherein said benzodiazepine is zolazepam or valium; said opioid is morphine, butorphanol or fentanyl; said α-2 adrenergic agonist is medetomidine or xylazine; said NSAID is etodolac, carprofen, deracoxib, firocoxib, tepoxalin, or meloxicam; said corticosteroid is cortisol; said barbiturate is phenobarbital or thiopental; said non-barbiturate hypnotic is etomidate or alphaxan; said channel-blocking NMDA antagonist is ketamine or tiletamine; and/or said injectable is propofol or alfaxan.

15. The method of claim 1, wherein said subject is a dog, cat, horse, cow, or pig.

16. The method of claim 1, wherein the general anesthetic is administered before administration of the NMDA glutamate receptor antagonist.

17. The method of claim 1, wherein the general anesthetic is administered during or after administration of the NMDA glutamate receptor antagonist.

18. A method for prolonging anesthesia in a subject, said method comprising, administering to said subject an NMDA glutamate receptor antagonist and a general anesthetic.

19. A kit comprising an NMDA glutamate receptor antagonist, a general anesthetic and instructions for anesthetizing a subject.

20. The kit of claim 19, wherein the general anesthetic is separate from the NMDA glutamate receptor antagonist.
